SEQUENCE LISTING (1 sequence)

SEQ ID NO: 1 - DNA, 3853 bp, Torque teno virus
  source 1..3853: mol_type = genomic DNA
attttgctacgtcactaaccacgtgacacccacaggccaaccgaatgctatgtcatccatttcctgggccgggtctacgtcctcatataagtaagtgcacttccgaatggctgagttttccacgcccgtccgcagcggtgaagccacggagggagatctccgcgtcccgagggcgggtgccgaaggtgagtttacacaccgaagtcaaggggcaattcgggctcgggactggccgggctatgggcaaggctctgaaaaaagcatgtttattggcaggcattacagaaagaaaagggcgctgtcactgtgtgctgtgcgaacaacaaagaaggcttgcaaactactaatagtaatgtggaccccacctcgcaatgatcaacactaccttaactggcaatggtactcaagtatacttagctcccacgctgctatgtgcgggtgtcccgacgctgtcgctcattttaatcatcttgcttctgtgcttcgtgccccgcaaaacccaccccctcccggtccccagcgaaacctgcccctccgacggctgccggctctcccggctgcgccagaggcgcccggagatagagcaccatggcctatggctggtggcgccgaaggagaagacggtggcgcaggtggagacgcagaccatggaggcgccgctggaggacccgaagacgcagacctgctagacgccgtggccgccgcagaaacgtaaggagacgccgcagaggagggaggtggaggaggagatataggagatggaaaagaaagggcaggcgcagaaaaaaagctaaaataataataagacaatggcaaccaaactacagaaggagatgtaacatagtaggctacatccctgtactaatatgtggcgaaaatactgtcagcagaaactatgccacacactcagacgataccaactacccaggaccctttggggggggtatgactacagacaaatttactttaagaattctgtatgacgagtacaaaaggtttatgaactactggacagcatctaacgaagacctagacctttgtagatatctaggagtaaacctgtactttttcagacacccagatgtagattttatcataaaaattaataccatgcctccttttctagacacagaactcacagcccctagcatacacccaggcatgctagccctagacaaaagagcaagatggatacctagcttaaaatctagaccgggaaaaaaacactatattaaaataagagtaggggcaccaagaatgttcactgataaatggtacccccaaacagatctttgtgacatggtgcttctaactgtctatgcaaccgcagcggatatgcaatatccgttcggctcaccactaactgactctgtggttgtgaacttccaggttctgcaatccatgtatgataaaacaattagcatattaccagacgaaaaatcacaaagagaaattctacttaacaagatagcaagttacattcccttttataataccacacaaactatagcccaattaaagccatttatagatgcaggcaatgtaacatcaggcgcaacagcaacaacatgggcatcatacataaacacaaccaaatttactacagcaaccacaacaacttatgcatatccaggcaccaacagacccccagtaactatgttaacctgtaatgactcctggtacagaggaacagtatataacacacaaattcaacagttaccaataaaagcagctaaattatacttagaggcaacaaaaaccttgctaggaaacaccttcacaaatgaggactacacactagaatatcatggaggactgtacagctcaatatggctatcccctggtagatcttactttgaaacaacaggagcatatacagacataaagtacaatccattcacagacagaggagaaggcaacatgttatggatagactggctaagcaaaaaaaacatgaactatgacaaagtacaaagtaaatgcttaatatcagacctacctctatgggcagcagcatatggatatgtagaattttgtgcaaaaagtacaggagaccaaaacatacacatgaatgccaggctactaataagaagtccctttacagacccacaactactagtacacacagaccccacaaaaggctttgttccttactctttaaactttggaaatggtaaaatgccaggaggtagtagtaatgtgcctattagaatgagagctaaatggtatccaacattatttcaccagcaagaagtactagaggccttagcacagtcaggcccctttgcataccactcagacattaaaaaagtatctctgggtatgaaataccgttttaagtggatctggggtggaaaccccgttcgccaacaggttgttagaaatccctgcaaagaaacccactcctcgggcaatagagtccctagaagcttacaaatcgttgacccgaaatacaactcaccggaactcacattccatacctgggacttcagacgtggcctctttggcccgaaagctattcagagaatgcaacaacaaccaacaactactgacattttttcagcaggccgcaagagacccaggagggacaccgaggtgtaccactccagccaagaaggggagcaaaaagaaagcttacttttccccccagtcaagctcctcagacgagtccccccgtgggaagactcgcagcaggaggaaagcgggtcgcaaagctcagaggaagagacgcagaccgtctcccagcagctcaagcagcagctgcagcaacagcgaatcctgggagtcaaactcagactcctgttcaaccaagtccaaaaaatccaacaaaatcaagatatcaaccctaccttgttaccaagggggggggatctagcatccttatttcaaatagcaccataaacatgtttggtgaccccaaaccttacaacccttccagtaatgactggaaagaggagtacgaggcctgtagaatatgggacagaccccccagaggcaacctaagagatacccctttctacccctgggcccccaaggaaaaccagtaccgtgtaaactttaaacttggattccaataaagctaggccgtgggactttcacttgtcggtgtctgcttataaaagtaactaagcactccgagcgaagcgaggagtgcgacccttgggggctcaacgccttcggagccgcgcgctacgccttcggctgcgcgcggcacctcagacccccgctcgtgctgacacgctcgcgcgtgtcagaccacttcgggctcgcgggggtcgggaaatttactaaacagactccgagttgccattggactcaggagctatgaatcagtaacgaaagtgagtggggccagacttcgccataaggcctttatcttcttgccatttgtcagtaacaggggtcgccatagacttcggcctccactttaccttgtaaaaactaccaaaatggccgttccagtgacgtcacagccgccattttaagtagctgacgtcaaggattgacgtaaaggttaaaggtcatcctcggcggaagctacacaaaatggtggacaacatcttccgggtcaaaggttgtgcgtacgtcacaagtcacgtggaggggacccgctgtaacccggaagtaggccccgtcacgtgacttaccacgtgtgtacacgtcaccgccgccattttgttttacaaaatggctgacttccttcctcttttttgaaaaaaggcgccaaaaaaccgtcggcgggggggccgcgcgctgcgcgcgcggcccccggggggaggcattgcctcccccccccgcgcgcatgcgcgcgggtccccccccctccggggggctccgccccccggcccccccc

(19) United States
(12) Patent Application Publication
(10) Pub. No.: US 2025/0092472 A1
(43) Pub. Date: Mar. 20, 2025

(54) USE OF TORQUE TENO VIRUS (TTV) AS A MARKER TO DETERMINE THE RISK OF COMPLICATIONS IN A PATIENT ADMITTED TO A HEALTHCARE FACILITY

(71) Applicants: BIOMERIEUX, Marcy L'Etoile (FR); BIOASTER, Lyon (FR); HOSPICES CIVILS DE LYON, Lyon (FR)

(72) Inventor: François MALLET, Villeurbanne (FR)

(73) Assignees: BIOMERIEUX, Marcy L'Etoile (FR); BIOASTER, Lyon (FR); HOSPICES CIVILS DE LYON, Lyon (FR)

(21) Appl. No.: 18/580,232

(22) PCT Filed: Jul. 19, 2022
(86) PCT No.: PCT/FR2022/051436

§ 371 Date: Jan. 18, 2024

(30) Foreign Application Priority Data
Jul. 19, 2021 (FR) FR2107771

(51) Int. Cl. C12Q 1/70

(52) U.S. Cl. CPC C12Q 1/701; C12Q 1/705; C12Q 2600/118; C12Q 2600/156

(57) ABSTRACT

A method for in vitro or ex vivo assessment of the risk of complications in a patient admitted to a healthcare facility includes measuring the viral load of at least one torque teno virus (TTV) in a biological sample of the patient, wherein the patient is not undergoing immunosuppressive treatment.

### CROSS-REFERENCE TO RELATED APPLICATIONS

This application is a national phase entry of PCT Patent Application Serial No. PCT/FR2022/051436, filed on Jul. 19, 2022, which claims priority to French Patent Application Serial No. FR2107771, filed on Jul. 19, 2021, both of which are incorporated by reference herein.

### BACKGROUND

Secondary infections (or healthcare-associated infections, HAIs) are major complications in medical treatment, in particular in medical care facilities such as hospitals, where they are referred to as nosocomial infections. Nosocomial infections occur in 20 to 40% of patients admitted to intensive care units and result in prolonging the hospital treatment, often combined with the use of invasive medical devices and the administration of larger amounts of antibiotics. Ultimately, morbidity and mortality are also higher.

The occurrence of healthcare-associated infections has been particularly exacerbated in the last few years due to the increase in multidrug-resistant pathogens. The World Health Organization (WHO) estimates the number of nosocomial infections in European hospitals to be approximately 5 million, leading to approximately 50 000 deaths and an annual additional cost of 13 to 24 billion euros. Recommendations have been published, and the setting up of infection control programs has been encouraged, in particular by the U.S. Department of Health and Human Services, the European Centre for Disease Prevention and Control, the WHO and national agencies, for which preventing and reducing healthcare-associated infections have become a top priority. According to some models, a test that would reduce the time taken to identify patients with a high risk of contracting healthcare-associated infections would make it possible to reduce mortality in these patients, and with good cost-effectiveness.

It is therefore important to be able to identify early markers associated with a HAI risk and more generally with a risk of developing complications during medical treatment, particularly in order to provide a suitable therapeutic response as soon as a risk is detected.

Numerous factors influence the occurrence and development of healthcare-associated infections, such as factors associated with the patient's treatment but also the patient's general state of health. Several studies have demonstrated the existence of a relationship between compromised immune systems and an increased incidence of secondary infections (review in Delano et al., 2016), however these results remain controversial and a more recent study questions the existence of a link (Venet et al., 2021).

Some studies have also attempted to establish a link between the phenomenon of virus reactivation (herpesvirus (Hpv) and torque teno virus (TTV) in particular) and the occurrence of healthcare-associated complications. The phenomenon of virus reactivation exists particularly in patients who are not immunosuppressed, are seriously ill (sepsis) or wounded (trauma, burns, surgery), who have immunoparalysis (characterized by dysfunction of the adaptive and innate immune systems in response to an initial hyperinflammatory state).

At present, the clinical implications of virus reactivation have not been clearly established. In particular, it is unclear if viruses that reactivate should be considered as simple markers that reflect an alteration in the immune system, or on the contrary as pathogens that promote secondary infections and require preventative treatment to be implemented (Limaye et al, 2010).

Recently, Mallet et al. (Mallet et al., 2019—Intensive Care Medicine Exp. 7:28) published the results of a study suggesting the existence of a link between the co-reactivation of EBV (Epstein-Barr virus) and TTV viruses, and a non-significant reduction in mortality.

Another recent study conducted in patients who had received a kidney transplant established that measuring the TTV viral load made it possible to identify those patients with a low risk of secondary infection (Strassl et al., The Journal of Infectious Diseases, 2018; 218:1191-9). Nevertheless, the scope of this study remains limited, all the more so since the patients involved had all received immunosuppressive treatment and were therefore at greater risk of developing infectious diseases. Therefore, this does not make it possible to establish a credible link between the TTV viral load and the risk of a secondary infection (or a healthcare-associated infection) and therefore to identify which patients have a high risk of developing a secondary infection.

There is therefore still a need for a simple and reliable diagnostic method that makes it possible to identify patients at risk of developing complications, in particular a healthcare-associated infection, when they are undergoing medical treatment in a healthcare facility.

### SUMMARY

A first subject of the present invention relates to a method for the in vitro or ex vivo evaluation of the risk of complications in a patient admitted to a healthcare facility, comprising measuring the viral load of at least one torque teno virus (TTV) in a biological sample from said patient, characterized in that said patient is not undergoing immunosuppressive treatment.

According to a preferred embodiment, said method is characterized in that the patient is a patient admitted to hospital, preferably to the emergency department, the resuscitation department, the intensive care unit or the high-dependency unit.

According to another preferred embodiment, said method is characterized in that said patient is a patient in a septic state, more particularly in septic shock, a patient suffering from burns, more particularly severe burns, a patient suffering from trauma, more particularly severe trauma, or a patient who has undergone a surgical operation, more particularly a major surgical operation.

According to another preferred embodiment, said method is characterized in that the biological sample originates from a patient in a septic state, suffering from burns, suffering from trauma, or who has undergone a surgical operation, and who has been admitted to the intensive care unit.

According to another preferred embodiment, said method is characterized in that the complications consist of the occurrence of a healthcare-associated infection (HAI).

According to another preferred embodiment, said method is characterized in that it comprises carrying out the steps consisting in:
a) measuring the viral load of at least one TTV in a biological sample from said patient,
b) comparing the viral load determined for said biological sample to a predetermined reference value, and
c) establishing a risk of complications when the viral load determined for said biological sample is greater than or less than the predetermined reference value.

According to another preferred embodiment, said method is characterized in that the predetermined reference value is 10 000 copies/ml.

According to another preferred embodiment, said method is characterized in that it comprises carrying out the steps consisting in:
a) measuring a first viral load of at least one TTV in said biological sample from the patient originating from a first sample taken at the time T1,
b) measuring a second viral load of at least one TTV in said biological sample from the patient originating from a second sample taken at the time T2,
c) calculating the variation between the viral load at T2 and the viral load at T1, giving a value ΔTTV,
d) drawing a conclusion regarding the risk of complications from the result of the comparisons.

According to a preferred embodiment, said method is characterized in that it comprises carrying out the steps consisting in:
a) measuring a first viral load of at least one TTV in said biological sample from the patient originating from a first sample taken at the time T1,
b) measuring a second viral load of at least one TTV in said biological sample from the patient originating from a second sample taken at the time T2,
c) calculating the variation between the viral load at T2 and the viral load at T1, giving a value ΔTTV,
d) drawing the conclusion that an increased risk of complications is absent when the value ΔTTV is within the range extending from −1.25 to +1.25, preferably from −1.20 to +1.20, and more preferably still from −1.10 to +1.10.

According to another preferred embodiment, said method is characterized in that it also comprises detecting the presence of at least one herpesvirus (HPV).

According to another preferred embodiment, said method is characterized in that it also comprises measuring the viral load of at least one herpesvirus (HPV).

According to another preferred embodiment, said at least one herpesvirus (HPV) is selected from the group consisting of: CMV, EBV, HHV6 and HSV-1, and is preferably EBV.

According to another preferred embodiment, said method is characterized in that the viral load is measured by amplification, sequencing or hybridization of at least one TTV sequence, and of at least one herpesvirus where appropriate, preferably by amplification, more preferentially by real-time PCR.

According to another preferred embodiment, said method is characterized in that the biological sample is a biological fluid from the patient, said fluid being selected from the group consisting of blood or derivatives thereof, such as plasma and/or serum, cerebrospinal fluid, urine, and bronchoalveolar lavage.

### BRIEF DESCRIPTION OF THE DRAWINGS

FIG. 1: Representation of the structure of a genome of a TTV isolate.

FIG. 2A and FIG. 2B: Characteristics of patients from the cohort on admission and results on the basis of the TTV viral titer, kinetics of viremia and concomitant infection with herpesviruses (FIGS. 2A and 2B). The categorical variables are expressed as n(%) and the continuous variables as median [Q1-Q3]. The comparisons between the TTV conditions regarding the titer or viremia kinetics or concomitant infection with herpesvirus were performed using a chi squared test for the qualitative variables and a Wilcoxon test for the quantitative variables, depending on the case. The P values in bold with asterisks represent significance at p<0.05. ICU=intensive care unit, HAI=infection acquired in ICU, HpV=herpesvirus, SOFA=Sequential Organ Failure Assessment, SAPS=Simplified Acute Physiology Score. a TTV DNAemia less than 10 000 copies/ml, b TTV DNAemia greater than 10 000 copies/ml, c Stability of TTV titer corresponding to a gradient within the range −1.25/+1.25 (measured between the medians of the viral titers at (D1 to D7) and (D14-D28)), d Increase in TTV titer corresponding to gradient >+1.5, e Decrease in TTV titer corresponding to gradient <−1.5 f TTV DNAemia greater than 10 000 copies/ml and at least one herpesvirus, g Exclusively TTV DNAemia greater than 10 000 copies/ml, h Exclusively herpesvirus (HpV).

FIG. 3: Association between the occurrence of an HAI during the month of hospitalization and the presence of viremia.

FIG. 4: Characteristics of patients of the cohort on admission and results on the basis of TTV DNAemia greater than 167 copies (cp)/ml (LOD), 7000 copies/ml, 10 000 copies/ml and 40 000 copies/ml, during the first month (D1 to D28) following admission.

FIG. 5: Association between the binary clinical result and the kinetics of TTV expression.

FIG. 6: Reference for the HpV viral strains.

FIG. 7: Reference table of PCR conditions for determining the LOD of the viruses.

### DETAILED DESCRIPTION

The subject of the present invention is a method for evaluating a risk of complications, for example a healthcare-associated infection (HAI), in a patient who is not taking an immunosuppressive treatment and who has been admitted to a health facility.

Indeed, the inventors have shown that the torque teno virus (TTV) viral load is an indicator of the risk of complications in patients who are not taking an immunosuppressive treatment and who have been admitted to a health facility, and more particularly in patients who are being treated in the intensive care unit.

In particular, it appears, surprisingly, that the increase in the TTV viral load after the first week following admission to intensive care is significantly correlated with a longer stay in this unit.

Moreover, both an increase and a decrease in TTV viral load are significantly associated with the occurrence of a HAI during the first month following admission.

Finally, the concomitant detection, from the first week following admission of the patient, particularly to the intensive care unit, of a high TTV viral load associated with the presence of at least one herpesvirus, is correlated with a risk of occurrence of a healthcare-associated infection (HAI). It is thus possible to make an early evaluation of the risk of occurrence of complications, and in particular the risk of occurrence of a healthcare-associated infection (HAI), in patients who are not undergoing immunosuppressive treatment.

In the present description, the embodiments can be taken alone or suitably combined by those skilled in the art.

### Methods for Evaluating the Risk of Complications in a Patient

The subject of the present description is a method for the in vitro or ex vivo evaluation of the risk of complications in a patient who is not taking an immunosuppressive treatment and who has been admitted to a healthcare facility, on the basis of a measurement of the viral load of at least one TTV in a biological sample from said patient.

According to a first aspect, a method for the in vitro or ex vivo evaluation of the risk of complications in a patient admitted to a healthcare facility is described herein, comprising measuring the viral load of at least one TTV in a biological sample from said patient, characterized in that said patient is not undergoing immunosuppressive treatment.

Surprisingly, by studying virus reactivation phenomena in patients in a healthcare facility, the inventors have identified, in patients who are not undergoing immunosuppressive treatment, the existence of a correlation between the change in the TTV viral load during their stay in the healthcare facility and the occurrence of complications, i.e. an unfavorable change in their condition, namely in particular the occurrence of a healthcare-associated infection.

According to a preferred embodiment, a variation in the TTV viral load during the first month following admission to the healthcare facility indicates a risk of developing a HAI.

The expressions “evaluating the risk of complications in a patient” or “determining a risk of complications in a patient” here denote the determination of the probability of a subject or patient developing complications, as defined below, in the future.

The methods disclosed herein represent tools for evaluating said risk, and can be combined with other processes, methods or indicators such as clinical examination, taking a biological sample from said patient and detecting one or more biomarkers and/or one or more infectious agents, particularly bacteria, viruses, parasites, yeasts or filamentous fungi.

“Complications” here means an unfavorable change in a disease, a state of health or a medical treatment. “Complications” here may in particular mean an unfavorable change in a disease which may require the implementation of one or more invasive medical devices, for example tracheal intubation, the insertion of a urinary catheter, a catheter or a central line.

“Complications” also means an unfavorable change in a disease which may result in prolonging treatment in said healthcare facility, in particular in the intensive care unit.

“Complications” also means the death of said patient.

“Complications” preferably means an unfavorable change in a disease which may require a specific medical treatment to be implemented. As an example of such complications, mention may be made of the development of a secondary infection or an opportunistic infection, in particular of a “healthcare-associated infection” or “HAI”, such as infection with one or more infectious agents, for example bacteria, viruses, parasites, yeasts or filamentous fungi.

In the context of the present description, an infection is said to be a “healthcare-associated infection” if it occurs during treatment (diagnostic, therapeutic, palliative, preventative, educational or surgical) of a patient by a healthcare professional, and if it was not present at the start of treatment. Healthcare-associated infections cover infections contracted in a healthcare facility (referred to as nosocomial infections) but also those contracted during care received outside of this context. When the state of infection is not known precisely at the start of treatment, a delay of at least 48 hours or a delay longer than the incubation period is commonly accepted in order to define a HAI.

For infections of surgical sites, those infections occurring in the 30 days following surgery or, if an implant, prosthesis or prosthetic material has been put in place, those infections occurring in the year following the surgery, are considered to be healthcare-associated infections. The healthcare-associated infection may be of bacterial origin, for example by a bacterium such as Escherichia coli, Staphylococcus aureus or Pseudomonas aeruginosa. The infection may also be fungal in origin, particularly by a yeast of the genus Candida, or a filamentous fungus of the genus Aspergillus, in particular Aspergillus fumigatus. The infection may also be viral, particularly by a virus of the adenovirus, herpesvirus, enterovirus, rotavirus or HIV type. This may also be the reactivation of potentially pathogenic latent viruses, such as the herpesvirus, for example the viruses CMV, EBV, HHV6 and HSV-1.

According to a preferred embodiment, the description relates to a method for the in vitro or ex vivo evaluation of the risk of complications in a patient who is not taking an immunosuppressive treatment, said complications being the occurrence of a healthcare-associated infection (HAI).

In particular, the HAI may be a nosocomial infection. In this case, the method described herein makes it possible to determine the risk of occurrence of a nosocomial infection in said patient. In the case of a patient in a septic state, who is therefore already suffering from a primary infection, the method described herein makes it possible to determine the risk of occurrence of a secondary infection.

The method described herein is based in particular on measuring the viral load of at least one TTV in a biological sample from said patient.

“Biological sample” means any sample which can be taken from a subject.

Generally, the biological sample must make it possible to determine the TTV and/or herpesvirus (HPV) load. The biological sample here means, inter alia, but without being limited thereto, whole blood, serum, plasma, expectorate, nasopharyngeal samples, urine, stools, skin, cerebrospinal fluid, saliva, gastric secretions, sperm, seminal fluid, tears, spinal tissue or fluid, cerebral fluid, a trigeminal ganglion sample, a sacral ganglion sample, adipose tissue, lymphoid tissue, placental tissue, upper reproductive tract tissue, gastrointestinal tract tissue, genital tissue and nervous system tissue.

In particular, this sample may be a biological fluid, such as a blood sample or a blood-derived sample, which may particularly be chosen from whole blood (as collected from the veins, i.e. containing white and red blood cells, platelets and plasma), plasma, serum, and also all types of cells extracted from the blood, such as peripheral blood mononuclear cells (PBMCs, containing lymphocytes (B, T and NK cells), dendritic cells and monocytes), B cell sub-populations, purified monocytes or neutrophils. The sample to be tested can be used directly from the biological source or following pretreatment that makes it possible to modify the nature of the sample. For example, such a pretreatment may include the preparation of plasma from blood, the dilution of viscous fluids, and so on.

Pretreatment methods may also involve the filtration, precipitation, dilution, distillation, mixing, concentrating, inactivating disruptive components, adding reagents, lysis, etc. In addition, it may be beneficial to modify a solid test sample to form a liquid medium or to release the analyte.

Preferably, the biological sample is a biological fluid from the patient, said fluid being selected from the group consisting of blood or derivatives thereof, such as plasma and/or serum, cerebrospinal fluid, urine and bronchoalveolar lavage. Even more preferably, the biological sample is a sample of blood or a derivative thereof, such as plasma and/or serum.

Here, the term “subject” refers to a vertebrate, preferably a mammal, and most preferably of all a human. A human subject may also be referred to as a “patient”.

“Patient” refers more particularly here to a human subject who has been in contact with a healthcare professional such as a doctor (for example a general practitioner) or a medical facility or health facility (for example a hospital and more particularly the emergency department, the resuscitation department, the intensive care unit or the high-dependency unit, or a medical facility for the elderly, of the assisted living type). According to some embodiments, the patient may be an elderly person in the context of a vaccination protocol (particularly in an assisted living facility or else at a general practitioner).

As described here, said evaluation method makes it possible to determine the risk of developing complications in a patient who is not undergoing immunosuppressive treatment.

“Patient who is not undergoing immunosuppressive treatment” means that said patient is not taking any immunosuppressive medicaments, or medicaments with immunosuppressive side effects, and who is also not undergoing any treatment that could induce immunosuppression.

“Immunosuppressive treatment” or “inducing immunosuppression” mean treatments, particularly medicaments, the effect of which is to reduce, inhibit or prevent the activity of the immune system. By way of nonlimiting example, an “immunosuppressive treatment” may particularly be a treatment that implements glucocorticoids, cytostatics, interferons, antiproliferative and antimetabolic medicaments (rapamycin, everolimus, mycophenolate mofetil, mycophenolic acid), calcineurin inhibitors, particularly for their anti-transplant-rejection action (cyclosporine, tacrolimus (FK506), voclosporine), S1P-R agonists (FTY720), malononitrilamides (FK778), antibodies, for instance antithymocyte globulin, or some monoclonal antibodies directed against specific antigens such as muromonab-CD3, daclizumab, basiliximab, rituximab, alemtuzumab, infliximab, adalimumab, efalizumab.

“Immunosuppressive treatment” also means radiotherapy or chemotherapy.

According to a preferred embodiment, the description relates to a method for the in vitro or ex vivo evaluation of the risk of complications in a patient who is not taking an immunosuppressive treatment and who has been admitted to a healthcare facility, in particular to the emergency department, the resuscitation department, the intensive care unit or the high-dependency unit.

A distinction is made between several departments within a hospital, depending on the patient's medical circumstances.

“Emergency department” means the department that receives ill and injured people who have either arrived by themselves or have been brought by ambulances or emergency services vehicles. The role of the emergency services is to receive indiscriminately, at all hours of the day and night, and all year round, any person in an emergency situation, including psychiatric emergencies, and to treat that person, particularly in the case of distress and life-threatening emergencies.

“Resuscitation department” means the specialist department where patients in the most serious conditions are hospitalized. In this department, patients benefit from constant monitoring of vital functions such as ventilation, oxygenation, blood pressure, cardiac and renal function. If required, these vital functions can be assisted in order, if possible, to allow the patient to survive. Patients are admitted to the resuscitation department if they present with failure of a vital function, for example during a serious infection (septic shock), drug intoxication, multiple trauma, coma, acute kidney failure, acute respiratory failure, after cardiac arrest or else in the post-operative phase following major surgery, such as heart surgery or surgery of the digestive tract.

“Intensive care unit”, “intensive care department” or “ICU” mean the department responsible for treating patients in critical conditions, i.e. presenting with failure of at least one vital function, or patients who are at risk of developing serious complications. The intensive care unit has highly specialist technical means. These means are continually used by a multidisciplinary team in order to detect, prevent and correct acute imbalances which are assumed to be reversible and which are associated with the underlying disorder (disease, surgery, trauma, intoxication, burns, sepsis).

“High-dependency unit” or “HDU” means a hospital department for receiving and treating ill people who require close monitoring. High dependency units treat patients whose condition and treatment suggest that one or more failures of vital functions might occur, and patients whose condition after recovering from one or more failures of vital functions (after a stay in the resuscitation department, for example) is too serious or unstable to enable them to return to a conventional hospital unit. High-dependency units are an intermediate level between resuscitation departments and conventional care departments.

According to a preferred embodiment, the present in vitro or ex vivo evaluation method makes it possible to evaluate the risk of complications in a patient who has been admitted to the intensive care unit.

The present evaluation method is more particularly suitable for evaluating the risk of complications in a patient who has suffered from an immune-inflammatory attack.

“Immune-inflammatory attack” means a trauma or a wound, the effect of which is to induce a hyperinflammatory response in the patient's body. The persistence of this hyperinflammatory response leads to acquired immunosuppression, associated with an anti-inflammatory profile, and ultimately to the inability to halt infections. This phenomenon, also known as immunoparalysis, is characterized by the downregulation of the expression of molecules of the major histocompatibility complex class II at the surface of monocytes but not B cells. Suppression of monocyte/macrophage function reduces the release of immune complexes, adversely affects antigen presentation ability, and reduces the function of natural killer (NK) cells. This type of reaction is common in non-immunosuppressed patients of intensive care units (Hotchkiss et al., 2013).

An “immune-inflammatory attack” includes in particular trauma, for patients suffering from trauma, burns, for patients suffering from burns, surgery, for patients who have undergone surgery, or sepsis for patients in a septic state.

According to a preferred embodiment, the description relates to an in vitro or ex vivo method for evaluating the risk of complications in a patient who is not taking an immunosuppressive treatment, said patient being in a septic state, more particularly in septic shock, and/or a patient suffering from burn(s), more particularly severe burns, and/or a patient suffering from trauma, more particularly severe trauma, and/or a patient who has undergone a surgical operation, more particularly a major surgical operation.

“Patient in a septic state” or “patient suffering from sepsis” means a patient presenting with at least one life-threatening organ failure caused by an inappropriate host response to infection.

“Septic shock” means a sub-type of sepsis in which hypotension persists despite adequate vascular filling.

“Patient suffering from burns” means that the patient has destruction of the skin cells and underlying tissue caused by a thermal burn and/or an electrical burn and/or a chemical burn and/or a radiation burn. A burn can be superficial, partial thickness or full thickness and be located generally or specifically, for example on the neck, face, eyes, hands, feet, joints or other parts of the body.

“Severe burn(s)” or “patient suffering from severe burn(s)” means a patient suffering from burns over a bodily surface area of greater than 15% of the total surface area of the body, preferably greater than 20%, preferably greater than 25% and most particularly greater than 30% of the total surface area of the body.

“Patient suffering from trauma” means a patient admitted directly to an intensive care unit. “Patient suffering from severe trauma” means a patient having an injury severity score (ISS, Baker et al, 1974) of greater than 15, preferably greater than 20 and more preferably still greater than 25.

“Patient who has undergone a surgical operation” means a patient who has undergone an invasive surgical procedure with the aim of treating a disease state such as a disease or wound, in order to aid or improve bodily functions, appearance, or to repair wounded areas.

“Major surgery” or “patient who has undergone major surgery” means a surgical procedure which is technically difficult and/or carries a risk of hemorrhage and/or carries a risk of death and/or which is long (for example more than 3 hours, preferably more than 4 hours, for instance 5 hours or 6 hours, or even longer) and/or which requires significant postoperative care. By way of nonlimiting example, major surgery means surgery intended for at least one of the following indications: esophagogastrectomy, Bricker bladder resection (total bladder resection with reconstruction from the small intestine), cephalic pancreaticoduodenectomy (Whipple procedure) and/or abdominal aortic aneurysm surgery by laparotomy.

Some preferred embodiments are described below. The present description is not limited to these embodiments and it will be possible to implement other particular embodiments by combining one or more of the features described above.

The inventors thus studied phenomena of virus reactivation in patients in a healthcare facility. More particularly, they measured the presence and/or variation in the TTV viral load in biological samples from said patients at different times in their treatment in a healthcare facility.

The inventors observed in particular that it was possible for the viral load to change over time, depending on the patients. In particular, they observed, surprisingly, that this change is indicative of a risk of occurrence of a healthcare-associated infection and/or a risk of prolonging the duration of treatment. Thus, the inventors have developed a method for the in vitro or ex vivo evaluation of the risk of complications in a patient admitted to a healthcare facility.

The method described herein is based on a measurement of the TTV viral load. “Torque teno virus” or “TTV” means here a virus of the Anelloviridae family. A TTV as intended here is a non-enveloped virus with a circular single-stranded DNA genome of negative polarity.

“TTV genome” refers here to all genomes of all Anelloviridae, and particularly the genomes of the genera Alphatorquevirus (TTV), Betatorquevirus (TTMV), and Gammatorquevirus (TTMDV), found in humans. By way of example, reference is made here to the genome of the torque teno virus prototype strain, TTV-1a. More specifically, an example of a TTV genome is a sequence, such as is for example represented by SEQ ID No 1 and for which the Genbank accession number is AB017610.

In 1997, using gene subtraction methods, a viral sequence fragment of approximately 500 base pairs (bp) was discovered in the serum of a Japanese patient presenting with post-transfusion hepatitis of indeterminate etiology (1-7). This clone, initially named N22 and not listed in the viral sequence databases at the time, was renamed TT virus (TTV) after the initials of the patient in whom it was discovered (Nishizawa et al., 1997). Additional studies subsequently showed that the TTV genome consists of a circular single-stranded DNA of negative polarity. This was the first virus with circular single-stranded DNA isolated in humans. The use of primers that make it possible to amplify the sequences in this non-coding region demonstrated a high prevalence of TTV in the global population (around 90%).

TTV produces chronic infections without clearly associated clinical signs. It is referred to as an apathogenic or orphan virus. Numerous studies have thus focused on the involvement of TTV in human disease, particularly certain liver diseases, without a clear role being identified for this virus.

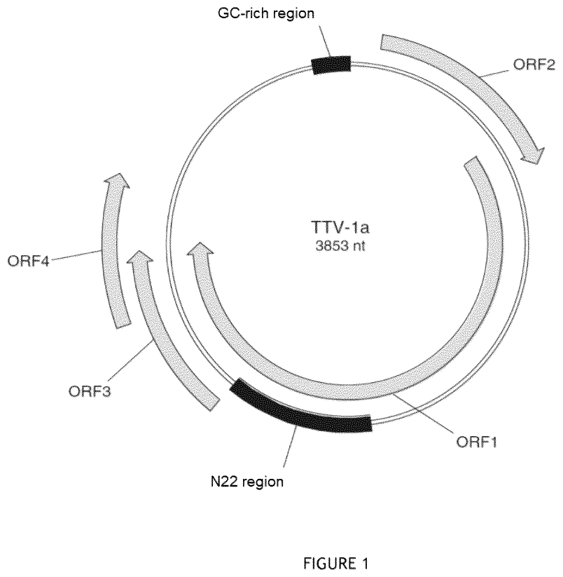

Preferentially, the genome of TTVs has a size of approximately 3.8 kb. The genomic structure and organization of TTVs is well known to those skilled in the art (cf. for example Biagini, Curr Top Microbiol Immunol. 331: 21-33, 2009) and is shown by way of example in [FIG. 1].

Thus, the TTV genome can be divided into an untranslated region (UTR) of approximately 1 to 1.2 kb and a potential coding region of approximately 2.6 to 2.8 kb. The coding region particularly contains two large open reading frames: ORF1 and ORF2, encoding two proteins of 770 and 202 residues, respectively. In the TTV genome represented by SEQ ID No 1, the open reading frames ORF1 and ORF2 are between nucleotides 589-2901 and 107-715, respectively. The TTV genome may have other open reading frames. For example, the genome of TTVs may comprise two additional open reading frames, ORF3 and ORF4 [FIG. 1].

The untranslated region UTR is highly conserved. It particularly comprises a GC-rich sequence liable to form a secondary structure. Amplification of sequences chosen in the UTR-5′ untranslated region made it possible to demonstrate that the prevalence of the virus is very high throughout the entire global population (Hu et al., J Clin Microbiol. 43(8): 3747-3754, 2005). This region particularly comprises a sequence of 128 bp and can be amplified using methods known to those skilled in the art, for instance the TTV R-GENE® diagnostic kit (bioMérieux, France).

“Viral load” means the number of virus particles present in a biological sample. Viral load reflects the seriousness of a viral infection. Viral load can be determined by measuring the amount of one of the components of the virus (genomic DNA, mRNA, protein, etc.) in the biological sample.

Preferably, viral load thus refers to the proportion of nucleic acid sequences that belong to said virus in a biological sample. More preferentially, viral load represents the number of copies of the genome of said virus in a biological sample.

In the present case, viral load represents the TTV load. “TTV load” corresponds here more particularly to the TTV viral load, i.e. to the number of particles of TTV virus present in a biological sample. The TTV load in a patient means the viral load of any TTV which said patient is hosting. The TTV load can be determined by measuring the amount of a component of TTV in this biological sample.

Preferably, the TTV load corresponds to the amount of TTV nucleic acid sequences present in a biological sample. Thus, the determination of the TTV load in a patient according to the protocol of the invention comprises estimating the number of sequences of all the TTVs in a biological sample from said patient. In particular, according to the method of the invention, there is no selection of specific TTV strains to be measured in said biological sample. Indeed, the detection of a high TTV viral load and particularly the detection of a variation in the TTV load is associated with an increased risk of complications, particularly of the occurrence of a healthcare-associated infection, independently of the Anelloviridae strain or strains detected.

Preferably, the determination of the TTV load comprises the determination of the amount of active and/or inactive virus copies. It consists in determining the amount of circulating, integrated or latent virus copies.

In particular, it is possible to determine the number of TTV virus copies by determining the TTV limit of detection beforehand.

“Limit of detection” or “LOD” means the smallest amount of copies of the genome that can be distinguished from an absence of detection (blank value) using viral standards. Next, by extrapolation from standard curves, it is then possible to determine a viral load in terms of copies of viral DNA per μl in the reaction tube, then in terms of copies of viral DNA per ml in said sample.

In the context of the present description, a viral load in the biological sample of greater than 7000 copies per ml of biological sample, for instance blood or a derivative thereof such as plasma and/or serum, preferably greater than 10 000 copies per ml of plasma, preferably greater than 20 000 copies per ml of biological sample, and more preferably still greater than 40 000 copies per ml of biological sample, is considered to be a high TTV viral load.

The TTV levels—and therefore the TTV load—can be determined using methods well known to those skilled in the art, by measuring levels of TTV DNA, TTV RNA or TTV proteins. The method according to the invention may thus comprise, between taking the sample from the patient and step a) as defined below, another preliminary step corresponding to converting the biological sample into a double-stranded DNA sample, or into an mRNA (or corresponding cDNA) sample, or into a protein sample, which is then ready to be used for the in vitro measurement of the TTV viral load in step a) as defined below.

The preparation or extraction of viral double-stranded DNA, mRNA (and also the reverse transcription thereof to give cDNA) or proteins from the cell sample are merely routine procedures that are known to those skilled in the art.

The double-stranded DNA can correspond either to the whole genome of the TTVs, or merely to a portion thereof. Once a double-stranded DNA, an mRNA (or a corresponding cDNA) or a ready-to-use protein sample is available, detection of the TTVs can be carried out, depending on the type of sample or conversion, either using genomic DNA (i.e. based on the presence of at least one sequence consisting of at least part of the TTV genome) or using mRNA (i.e. based on the content of TTV mRNA in the sample) or using protein (i.e. based on the content of TTV proteins in the sample).

The TTV levels are preferably determined by measuring the levels of TTV nucleic acid, more preferably TTV DNA.

Methods for detecting a nucleic acid in a biological sample are well known to those skilled in the art and comprise, inter alia, amplification, preferably amplification by PCR, more preferably by real-time PCR, sequencing, and hybridization with a labeled probe.

Advantageously, the description therefore relates to the method for the in vitro or ex vivo evaluation of the risk of complications in a patient who is not taking an immunosuppressive treatment and who has been admitted to a healthcare facility, comprising measuring the viral load of at least one torque teno virus (TTV) in a biological sample from said patient, said viral load being measured by amplification, sequencing or hybridization of at least one TTV sequence, preferably by amplification, more preferentially by real-time PCR.

According to a first embodiment, the TTV load is determined by amplification of the TTV sequences.

According to this embodiment, a preferred approach consists in amplifying sequences which are known to be specific to the genome of TTVs, and particularly preferably at least one sequence from the untranslated region, UTR, of TTVs, as defined previously.

“Sequence specific to the genome of TTVs” means here a sequence which is present in the majority of known TTVs but which is absent from the majority of other viruses, particularly from the majority of other anelloviruses. Preferably, a TTV-specific sequence is present in at least 90%, at least 95%, at least 96%, at least 97%, at least 98% or at least 99% of known TTV genomes. More preferably, it is present in 100% of known TTV genomes. Alternatively, a TTV-specific sequence is present in less than 10%, less than 5%, less than 4%, less than 3%, less than 2%, less than 1% of known genomes of anelloviruses other than TTVs. Preferably, a TTV-specific sequence is absent from all known genomes of anelloviruses other than TTVs. Particularly preferably, such a sequence is for example a sequence contained in the untranslated region, UTR, which is highly conserved from one TTV to another. More particularly, such a sequence can correspond to the 128 bp sequence of the 5′-UTR untranslated region, which can be amplified according to methods known to those skilled in the art, for example using the TTV R-GENE® diagnostic kit (bioMérieux, France).

Thus, according to this embodiment, the method described herein comprises the use of primers and probes for the amplification of sequences known to be specific to the genome of TTVs. As is customary in this technical field, these primers are preferably oligonucleotides. For example, these primers may comprise less than 30 nucleotides, less than 25 nucleotides, less than 20 nucleotides, less than 15 nucleotides or less than 12 nucleotides. Alternatively, these primers comprise at least 12, 15, 20, 25 or 30 nucleotides. Preferably, the primers used comprise between 12 and 20 nucleotides, between 12 and 25 nucleotides, between 15 and 20 nucleotides or else between 15 and 25 nucleotides. Those skilled in the art know how to determine the length and the sequence of the primers to be used to amplify the TTV-specific sequence(s). They may for example use the same primers as those provided in the TTV R-GENE® diagnostic kit (ref. 423414 and 69-030B, bioMérieux, France).

Amplification techniques comprise in particular isothermal techniques and techniques based on PCR (polymerase chain reaction). Isothermal amplification methods cover a large number of methods. The most commonly used to detect pathogens are the LAMP (loop-mediated amplification) and RPA (recombinase polymerase amplification) methods. Isothermal amplification methods also comprise methods, for instance NASBA (nucleic acid sequence-based amplification), HDA (helicase-dependent amplification), RCA (rolling circle amplification) and SDA (strand displacement amplification), EXPAR (exponential amplification reaction), ICANs (isothermal and chimeric primer-initiated amplification of nucleic acids), SMART (signal-mediated amplification of RNA technology), etc. (see for example Asiello et Baeumner, Lab Chip 11(8): 1420-1430, 2011).

Preferably, the PCR technique used quantitatively measures the initial amounts of DNA, cDNA or RNA. Examples of techniques based on PCR which can be used in the methods described here comprise, without being limited thereto, techniques such as real-time PCR (Q-PCR), reverse transcription PCR (RT-PCR), multiplex reverse transcription PCR, real-time reverse transcription PCR (QRT-PCR) and digital PCR. These techniques are well-known and readily available technologies for those skilled in the art.

Preferably, the TTV load is determined using real-time quantitative PCR. Numerous methods for detecting and quantifying TTVs have been described in the art (see for example Maggi et al., J Virol. 77(4): 2418-2425, 2003).

Reference will particularly be made to the method described by Kulifaj et al. (J Clin Virol. 105:118-127, 2018). This method is particularly advantageous because of its simplicity and robustness. It is based on amplification of a sequence contained in the non-coding UTR region. This sequence is present in all known TTVs, thereby giving the method a very high degree of specificity. In addition, it is particularly versatile and can be implemented using any type of PCR platform. It is particularly advantageous to use the TTV R-GENE® diagnostic kit (ref. 423414 and 69-030B bioMérieux, France) to implement this method.

Alternatively, the viral load is determined using digital PCR. Digital PCR involves several PCR analyses on extremely diluted nucleic acids, such that the majority of positive amplifications reflect the signal from a single template molecule. Digital PCR thus enables individual model molecules to be counted. The proportion of positive amplifications among the total number of PCRs analyzed enables the concentration of template in the original, or undiluted, sample to be estimated. This technique was proposed in order to enable a variety of genetic phenomena to be detected (Vogelstein et al., Proc Natl Acad Sci USA 96: 9236-924, 1999). Digital PCR, like real-time PCR, potentially makes it possible to make a distinction between very small quantitative differences in target sequences between samples.

According to another embodiment, the TTV DNA levels are measured by sequencing. As used herein, the term “sequencing” has its broadest accepted meaning, and refers to any technique known to those skilled in the art that makes it possible to determine the sequence of a polynucleotide molecule (DNA or RNA), i.e. to determine the sequence of nucleotides that make up this molecule.

The TTV DNA can thus be sequences by any technique known in the art. Sequencing as it is meant here comprises, inter alia, Sanger sequencing, whole genome sequencing, sequencing by hybridization, pyrosequencing (particularly 454 sequencing, Solexa Genome Analyzer sequencing), sequencing using capillary electrophoresis, cycle sequencing, single-base extension sequencing, solid-phase sequencing, high-throughput sequencing, massively parallel signature sequencing (MPSS), sequencing by reversible dye terminator, mate pair sequencing, short-read sequencing, sequencing with exonucleases, sequencing by ligation, single molecule sequencing, sequencing by synthesis, sequencing by electron microscopy, real-time sequencing, inverted termination sequencing, nanopore sequencing, reversible terminator sequencing, sequencing by semiconductor, SOLiD® sequencing, SMRT (Single Molecule Real-Time Analysis) sequencing, MS-PET sequencing, mass spectrometry, and combinations thereof. A particular embodiment uses high-throughput DNA sequencing by means, for example, of the platforms MiSeq, NextSeq 500, and the HiSeq series developed by llumina (Reuter et al., Mol Cell, 58: 586-597, 2015; Bentley et al. Nature; 456: 53-59, 2008), the 454 Genome Sequencer and Roche (Margulies et al. Nature; 437: 376-380, 2005), the SOLiD platform from Applied Biosystems (McKernan et al., Genome Res; 19: 1527-1541, 2009), the Polanator platform (Shendure et al., Science, 309: 1728-1732) or else the Helicos single molecule sequencing platform (Harris et al. Science; 320: 106-109, 2008). High-throughput sequencing also includes methods such as SMRT real-time sequencing (Roads et al., Genomics, Proteomics & Bioinformatics, 13(5): 278-289, 2015), Ion Torrent sequencing (WO 2010/008480; Rothberg et al., Nature, 475: 348-352, 2011) and nanopore sequencing (Clarke J et al. Nat Nanotechnol: 4: 265-270, 2009).

The sequencing is carried out on all the DNA contained in the biological sample, or on parts of the DNA contained in the biological sample. It is immediately apparent to those skilled in the art that said sample contains at least a mixture of TTV DNA and of host subject DNA. Moreover, the TTV DNA will probably only represent a minor fraction of the total DNA present in the sample. Advantageously, the DNA is randomly fragmented, generally using physical methods, prior to sequencing.

A first approach consists in sequencing specific sequences of the genome of a TTV species. Another approach consists in using a method that enables the quantitative genotyping of nucleic acids obtained from the biological sample with a high degree of precision. In a particular embodiment, the precision is obtained by analyzing a large number (for example millions or billions) of nucleic acid molecules without any amplification, using protocols based on prior knowledge of the target sequences (i.e. in this case, the TTV sequences).

In a preferred embodiment, the method of the invention comprises a step of quantifying the number of reads.

In a particular embodiment, a random sub-set of nucleic acid molecules from the biological sample is subjected to high-throughput sequencing. Preferably, the TTV sequences are identified in the overall sequencing data by comparison with publicly deposited TTV sequences. This comparison is advantageously based on the level of sequence identity with a known TTV sequence and makes it possible to detect even far-removed variants. Currently available software such as BLAST can be used to determine the level of identity between the sequences.

Thus, a sequence having at least 80%, at least 85%, at least 90%, at least 95%, at least 96%, at least 97%, at least 98%, at least 99% or 100% identity with a known TTV sequence is identified as being a TTV sequence. According to this embodiment, the determination of the TTV load therefore comprises numbering the TTV sequences identified by sequencing in the biological sample from the subject.

In another embodiment, the TTV load is determined by measuring the amount of a viral protein in the biological sample. It is thus possible to employ specific antibodies, in particular in well known technologies such as immunoprecipitation, immunohistology, Western blot, dot blot, ELISA or ELISPOR, electrochemiluminescence (ECLIA), protein chips, antibody chips, or tissue chips coupled with immunohistochemistry. The other techniques which may be used include the FRET or BRET techniques, microscopy or histochemistry methods, including in particular confocal microscopy and electron microscopy methods, methods based on the use of one or more excitation wavelengths and a suitable optical method, such as an electrochemical method (voltammetry and amperometry techniques), atomic force microscopy, and radiofrequency methods such as multipolar resonance spectroscopy, confocal and non-confocal, detection of fluorescence, luminescence, chemiluminescence, absorbance, reflectance, transmittance and birefringence or refractive index (for example surface plasmon resonance, ellipsometry, the resonant mirror method, etc.), flow cytometry, radioisotope or magnetic resonance imaging, analysis by polyacrylamide gel electrophoresis (SDS-PAGE), by mass spectrometry and liquid chromatography coupled with mass spectrometry (LC-MS/MS). All these techniques are well known to those skilled in the art, and it is not necessary to describe them in detail here. In addition, TTV protein-specific antibodies are already available.

In a preferred embodiment, the method described herein comprises an additional step of standardizing the amount of viral nucleic acid or protein measured.

According to a preferred embodiment, it may be advantageous to standardize the TTV levels, i.e. the amount of TTV DNA, TTV RNA or TTV proteins present in the biological sample, to a parameter specific to this sample.

The standardization of the measured TTV load to a specific parameter makes it possible to reduce the error rate during the comparison between viral loads of two different biological samples. An example of a parameter which may be useful for this standardization may be a physical parameter, independent of the content of the sample, for example the volume of said sample. It is also possible to standardize the amount of TTV DNA, TTV RNA or TTV proteins to the total amount of DNA, RNA or proteins present in the sample. Alternatively, it is possible to use a particular DNA or RNA sequence or a particular protein as a standardization tool. For example, this sequence or this protein may be a human sequence or protein.

Alternatively, the amount of TTV DNA or RNA or TTV proteins in a given sample is compared to an internal control.

To this end, the amount of TTV nucleic acid or protein measured in the biological sample can be correlated to a defined amount of a suitable nucleic acid or protein which can be identified and quantified, for instance a host or exogenous nucleic acid or protein. Preferably, this identifiable and quantifiable nucleic acid or protein is processed (for example amplified, sequenced, etc.) like the target nucleic acid or protein.

It is thus possible to add to the sample, from the start, a known amount of this identifiable and quantifiable nucleic acid or protein, which will subsequently be processed like the target nucleic acid or protein and will pass through all the sample preparation steps before the amount of this viral nucleic acid or protein is measured. The preparation steps may comprise means to protect the viral nucleic acid and to destroy the host nucleic acids, for example using different nucleases.

Alternatively, these steps may comprise means to protect the viral proteins and to destroy the host proteins, for example using different proteases. The internal control makes it possible to evaluate the quality and extent of the treatment (for example amplification or sequencing) of the molecules in question (nucleic acids or proteins) in the sample. Preferably, said internal control is a nucleic acid molecule having a known sequence, this nucleic acid molecule being present in the sample at a known concentration. More preferably, this nucleic acid molecule is the circular single-stranded genomic DNA molecule of a virus of known sequence and concentration in the sample. Such a known virus may for example be a virus of the Circoviridae family. The ratio of the number of sequences in the sample to the control makes it possible to estimate the absolute number of TTV genomes of known sequence and concentration. Alternatively, this internal control is a protein of known sequence which is present in the sample at a known concentration.

Once the TTV load is determined by measuring the amount of determined TTV nucleic acid or protein, the latter optionally being standardized, it may be advantageous to compare it to a predetermined reference TTV load.

According to this preferred embodiment, the description relates to an in vitro or ex vivo method for evaluating the risk of complications in a patient who is not taking an immunosuppressive treatment and who has been admitted to a healthcare facility, comprising carrying out the steps consisting in:
a) measuring the viral load of at least one TTV in a biological sample from said patient,
b) comparing the viral load determined for said biological sample to a predetermined reference value, and
c) establishing a risk of complications when the viral load determined for said biological sample is greater than or less than the predetermined reference value.

For the purposes of the present application, “reference TTV load” or “reference viral load” means any TTV load used as a reference.

This means that the reference TTV load corresponds to “a reference level of TTV nucleic acid (or protein)” or “a control level of TTV nucleic acid (or protein),” i.e. to a concentration of a TTV nucleic acid (or protein) used as a reference.

“A reference concentration of TTV nucleic acid (or protein)” is intended here to mean a base level measured in a control sample which is comparable to the tested sample and which is obtained from a subject or a group of subjects presenting with a particular trauma and/or infectious condition. This may for example be a subject or group of subjects who are healthy or who do not present with a particular trauma and/or infection. This may also be a patient or group of patients admitted to a healthcare facility, in particular to the intensive care unit, particularly a patient or group of patients who have suffered an immune-inflammatory attack, for instance a patient or group of patients suffering from sepsis, suffering from burn(s), trauma and/or who have undergone surgery. Finally, this may be the same individual who has undergone surgery, for example before or just after said surgery. The reference level may be determined by a plurality of methods. For example, the control may be a predetermined value that can be in various forms. It may be a single threshold value such as a median or mean. The “reference level” may be a single value that is also applicable to each patient individually. Alternatively, the reference level may vary on the basis of the specific sub-populations of patients. Thus, for example, older men could have a different reference level to that of younger men for the TTV load, and women could have a different reference level to that of men for this viral load. Furthermore, the “reference level” can be established on the basis of comparative groups, such as groups that do not have a high level of TTV nucleic acid (or protein) and groups that have high levels of TTV nucleic acid (or protein). Another example of comparative groups would be groups suffering from a particular disease, condition or symptoms, and disease-free groups. The predetermined value can be defined, for example, when a tested population is evenly (or unevenly) split into groups, such as a low-risk group, a medium-risk group and a high-risk group.

The reference level can also be determined by comparing the level of TTV nucleic acid (or protein) in populations of patients suffering from diseases or taking a treatment that leads to an immunosuppressed state. This can be accomplished for example by analysis by histogram, in which an entire cohort of subjects is presented in the form of a graph, with a first axis representing the level of said TTV nucleic acid (or protein) and a second axis representing the number of patients in the group of patients expressing the TTV nucleic acid (or protein) at a given level. Two discrete groups of subjects, or more, can be determined by identifying sub-populations of the cohort who have identical or similar TTV nucleic acid (or protein) levels. The reference level can then be determined on the basis of a level which best makes a distinction between these discrete groups. A reference level can also represent the levels of two or more of the TTV nucleic acids (or proteins) present. Two or more markers may be represented, for example, by a ratio of values for the levels of each marker.

Similarly, a population that appears to be in good health would have a “normal” range that differs from that of a population known to have a condition associated with a high concentration of said TTV nucleic acid (or protein). Consequently, the predetermined value selected can take into account the category within which an individual falls. Suitable ranges and categories can be selected simply by means of routine experimentation by those skilled in the art. “High” and “increased” mean higher than a selected control. Generally, the control will be based on normal individuals who appear to be in good health and within a suitable age range.

In a preferred embodiment, the reference concentration corresponds to the concentration of TTV nucleic acid (or protein) or of the combination of TTV nucleic acids (or proteins) in the general population.

It will also be understood that the controls in the method described herein can be, aside from predetermined values, biological samples measured in parallel with the tested samples. According to this embodiment, the reference level will be that of the TTV nucleic acid(s) (or protein(s)) in a sample from a subject in good health.

Preferably, the reference concentration of TTV nucleic acid (or protein) is the concentration of this TTV nucleic acid (or protein) in a subject in good health or in a population of subjects in good health.

According to another preferred embodiment, the reference concentration of the TTV nucleic acid (or protein) is the concentration of this TTV nucleic acid (or protein) in a patient who has suffered an immune-inflammatory attack or in a group of patients who have suffered an immune-inflammatory attack.

According to another preferred embodiment, the reference concentration of the TTV nucleic acid (or protein) will be the concentration of this TTV nucleic acid (or protein) in a patient suffering from sepsis or in a group of patients suffering from sepsis.

According to another preferred embodiment, the reference concentration of the TTV nucleic acid (or protein) is the concentration of this TTV nucleic acid (or protein) in a patient suffering from burn(s) or in a group of patients suffering from burn(s).

According to another preferred embodiment, the reference concentration of the TTV nucleic acid (or protein) is the concentration of this TTV nucleic acid (or protein) in a patient suffering from trauma or in a group of patients suffering from trauma.

According to another preferred embodiment, the reference concentration of the TTV nucleic acid (or protein) is the concentration of this TTV nucleic acid (or protein) in a patient who has undergone surgery or in a group of patients who have undergone surgery.

According to another preferred embodiment, the reference concentration of the TTV nucleic acid (or protein) is the concentration of this TTV nucleic acid (or protein) in the same individual who has undergone surgery at a specific point in time, for example before or just after said surgery.

By comparing the TTV viral load in the biological sample from said patient to a predetermined reference value, it is then possible to establish the risk of complications in said patient.

According to a particular embodiment, the predetermined reference value corresponds to a viral load in the biological sample from the patient, for example blood or a derivative thereof such as plasma and/or serum, of greater than 7000 copies per ml of biological sample, for example greater than 10 000 copies, 20 000 copies or else 40 000 copies per ml of biological sample. According to this particular embodiment, a TTV viral load in the patient that is greater than the predetermined reference value is correlated with the severity of the patient's condition and can also be correlated with an increased risk of complications, particularly of the occurrence of a healthcare-associated infection.

According to a variant of this particular embodiment, the predetermined reference value corresponds to a TTV viral load of 10 000 copies per ml of biological sample. Thus, according to this variant, the presence of an increased risk of complications is established when the TTV viral load determined in the biological sample from the patient is greater than said predetermined reference value. Conversely, the absence of an increased risk of complications is established when the TTV viral load determined in the biological sample from the patient is less than said predetermined reference value.

A greater TTV load in the patient compared to that of an individual or group of individuals in good health may be an indicator that there is an increased risk of complications in said patient.

The term “greater than” as used herein in some embodiments means a larger amount, for example an amount slightly greater than the original or reference amount, or for example an amount that greatly exceeds the original or reference amount, and particularly all the amounts in the range. As a variant, “increase” may refer to an amount or an activity which is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 80%, 90% or 100% more than the amount or activity with which the increased amount or activity is compared.

The term “less than” as used herein in some embodiments means a smaller amount, for example an amount slightly less than the original or reference amount, or for example an amount that is very much less than the original or reference amount, and particularly all the amounts in the range. As a variant, “decrease” may refer to an amount or an activity which is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 80%, 90% or 100% less than the amount or activity with which the reduced amount or activity is compared.

Advantageously, it is also possible to use, as reference TTV viral load, the viral load of at least one TTV in the same patient at a second point in time.

According to a preferred embodiment, the description relates to an in vitro or ex vivo method for evaluating the risk of complications in a patient who is not taking an immunosuppressive treatment and who has been admitted to a healthcare facility, comprising carrying out the steps consisting in:
a) measuring a first viral load of at least one TTV in said biological sample from the patient originating from a first sample taken at the time T1,
b) measuring a second viral load of at least one TTV in said biological sample from the patient originating from a second sample taken at the time T2,
c) calculating the variation between the viral load at T2 and the viral load at T1, giving a value ΔTTV,
d) drawing a conclusion regarding the risk of complications from the result of the comparisons.

Surprisingly, the inventors have determined that a variation in the TTV viral load in the patient during their stay in a healthcare facility, namely an increase or a decrease in the TTV viral load according to the values defined below, is correlated with an increased risk of complications in said patient, in particular an increased risk of developing a healthcare-associated infection.

This method is thus particularly useful for evaluating and monitoring, over time, the risk of complications in a patient admitted to a healthcare facility. In a preferred embodiment, the first sample in step a) is taken at a time T1 chosen from: the day of admission, the 1st day, the 2nd day, the 3rd day, the 4th day, the 5th day, the 6th day and the 7th day, it being understood that the first day corresponds to the day after admission and/or to the day after the day on which said patient suffered an immune-inflammatory attack.

In a particular embodiment, when said patient is a patient admitted to a healthcare facility for surgery, more particularly for major surgery, the first sample in step a) can be taken at the time of the surgery, i.e. just before, just after and/or during the surgical procedure.

In a preferred embodiment, the second sample of step b) is taken at a time T2, this second time being later than the first time T1 of step a). Preferably, the second sample is taken between the 8th day and the 31st day, and more preferably still between the 14th day and the 31st day. Thus, the time T2 is preferably chosen from: the 14th day, the 15th day, the 16th day, the 17th day, the 18th day, the 19th day, the 20th day, the 21st day, the 22nd day, the 23rd day, the 24th day, the 25th day, the 26th day, the 27th day, the 28th day, the 29th day, the 30th day and the 31st day.

In another embodiment, the second sample is taken after the 31st day, for instance at least 40 days, 60 days, 90 days, 100 days, 120 days, 150 days, 180 days, 210 days, 240 days, 270 days, 300 days, 330 days, 360 days after the admission of said patient to a healthcare facility.

In some embodiments, the TTV viral load is also measured on a plurality of samples taken at different times between the times T1 and T2. In particular, the TTV viral load is also measured on at least 1 sample, preferably at least 2 samples, preferably at least 3 samples, preferably at least 4 samples, preferably at least 5 samples, for instance 6, 7 or 8 samples at different times between the times T1 and T2.

According to some embodiments, the TTV viral load at the time T1 corresponds to the mean or median of the viral loads measured in a plurality of biological samples from said patient which were taken between the day of admission, and/or the day of the immune-inflammatory attack, and the 7th day. Preferably, the TTV viral load at the time T1 corresponds to the mean or median of the viral loads measured in at least two, preferably at least three, preferably at least four biological samples from said patient which were taken between the day of admission, and/or the day of the immune-inflammatory attack, and the 7th day. By way of nonlimiting example, the TTV viral load at the time T1 can correspond to the mean or median of the viral loads measured in the biological samples from said patient which were taken on days 1 or 2 and on days 3 or 4 and on days 5, 6 or 7.

According to some embodiments, the TTV viral load at the time T2 corresponds to the mean or median of the viral loads measured in a plurality of biological samples from said patient which were taken between the 8th day following admission, and/or following the immune-inflammatory attack, and the 31st day. Preferably, the TTV viral load at the time T2 corresponds to the mean or median of the viral loads measured in at least two, preferably at least three, preferably at least four biological samples from said patient which were taken between the 8th day following admission, and/or following the immune-inflammatory attack, and the 31st day. By way of nonlimiting example, the TTV viral load at the time T2 may correspond to the mean or median of the viral loads measured in biological samples from said patient which were taken on the 14th day, or on any day between the 13th day and the 18th day, and on the 28th day, or on any day between the 26th day and the 36th day.

Advantageously, it is then possible to calculate, in step c), the ATTV value corresponding to the variation between the TTV viral load at the time T2 and the viral load at the time T1, i.e.:

ΔTTV=(TTV viral load at time T2)−(TTV viral load at time T1)

On the basis of the TTV viral load at the time T1 and the TTV viral load at the time T2, those skilled in the art are able to calculate the gradient of the line defined by these two measurements. ΔTTV thus corresponds to the value of the gradient between the times T1 and T2.

According to the present description, the TTV viral load between the times T1 and T2 will preferably be considered to be stable if the ΔTTV value is within the range extending from −1.25 to +1.25, preferably within the range extending from −1.20 to +1.20, preferably within the range extending from −1.15 to +1.15, preferably within the range extending from −1.10 to +1.10. For example, stability of the viral load can correspond to a gradient within the range from −1.25 to +1.25, measured between the medians of the viral loads for a plurality of samples at T1 and T2.

In addition, the TTV viral load between the times T1 and T2 will preferably be considered to have increased if the ΔTTV value is greater than +1.25, preferably greater than +1.35, preferably greater than +1.40, preferably greater than +1.45, and more preferably still greater than +1.50.

In addition, the TTV viral load between the times T1 and T2 will preferably be considered to have decreased if the ΔTTV value is less than −1.25, preferably less than −1.35, preferably less than −1.40, preferably less than −1.45, and more preferably still less than −1.50.

Consequently, if the variation in TTV viral load between the times T1 and T2 (ΔTTV) is within the range extending from −1.25 to +1.25, it is possible to conclude in step d) that the patient does not have an increased risk of the occurrence of a healthcare-associated infection.

Conversely, if the variation in TTV viral load between the times T1 and T2 (ΔTTV) is not within the range extending from −1.25 to +1.25, it is possible to conclude in step d) that the patient has an increased risk of the occurrence of a healthcare-associated infection.

Thus, in a preferred embodiment, the method according to the present description comprises carrying out the steps consisting in:
a) measuring a first viral load of at least one TTV in said biological sample from the patient originating from a first sample taken at the time T1,
b) measuring a second viral load of at least one TTV in said biological sample from the patient originating from a second sample taken at the time T2,
c) calculating the variation between the viral load at T2 and the viral load at T1, giving a value ΔTTV,
d) drawing the conclusion that an increased risk of complications is absent when the value ΔTTV is within the range extending from −1.25 to +1.25, preferably from −1.20 to +1.20, and more preferably still from −1.10 to +1.10.

According to a particularly advantageous embodiment, the description relates to an in vitro or ex vivo method for evaluating the risk of complications in a patient who is not taking an immunosuppressive treatment and who has been admitted to a healthcare facility, comprising measuring the viral load of at least one torque teno virus (TTV) in a biological sample from said patient, and further comprising detecting the presence of at least one herpesvirus (HPV) in said biological sample from said patient.

Indeed, it is to the inventors' credit to have determined that the concomitant detection of a high viral load of TTV and of at least one HPV in the biological sample from the patient, particularly during the first week following their admission to the intensive care unit, is indicative of an increased risk of complications in said patient, in particular of an increased risk of developing a healthcare-associated infection. The link between early viremia (as early as the first week following admission) for TTV/HPV and the existence of an increased risk of developing a HAI during the first month is also particularly surprising.

The term “determining the presence of at least one herpesvirus” or “detecting at least on herpesvirus” as used herein covers qualitative and/or quantitative detection. In various embodiments, HPV is detected by detecting the levels of HPV nucleic acid, more preferably HPV DNA.

According to the present description, a herpesvirus is determined to be present or detected if at least one protein from at least one HPV strain, more preferably at least one antigen from at least one HPV strain, is detected in at least one biological sample from said patient.

Preferably, a herpesvirus is determined to be present or detected if at least one nucleic acid from at least one HPV strain, more preferably at least one DNA from at least one HPV strain, is detected in at least one biological sample from said patient. In this case, reference is made to positive viremia or positive DNAemia.

“Viremia” here means the content of viral particles, or the viral load, in a biological sample for a given virus. Viremia is positive when the number of viral particles detected is greater than a predefined threshold (LOD) in said biological sample for a given virus. Conversely, viremia is negative when said viral particles are not detected in said biological sample.

“DNAemia” means the detection of viral DNA in a biological sample, particularly in a sample of plasma, whole blood and/or leukocytes isolated from peripheral blood and/or in a buffy coat sample (the fraction of a blood sample that is not coagulated after centrifugation, which contains most of the white blood cells and platelets). Several techniques are available for detecting DNAemia, particularly techniques based on PCR, the hybrid capture and analysis of branched-chain DNA.

When DNA from the virus being studied is detected in said biological sample, DNAemia is positive. Conversely, DNAemia is negative when said viral DNA is not detected in said biological sample. “Viremic event” means that a given virus has been detected at least once during the study, and particularly that positive DNAemia for a given virus has been detected in at least one biological sample from the patient during the study.

According to this preferred embodiment, the patient who is not taking an immunosuppressive treatment has an increased risk of complications when the TTV viral load in a biological sample from said patient, for example blood or a derivative thereof such as plasma and/or serum, is greater than 7000 copies per ml of biological sample, preferably greater than 10 000 copies per ml of biological sample, and when the presence of herpesvirus is also detected.

According to this preferred embodiment, the method may thus comprise carrying out the steps consisting in:
a) measuring the viral load of at least one TTV in a biological sample from said patient,
b) comparing the viral load determined for said biological sample to a predetermined reference value of 10 000 copies/ml,
c) determining the presence of at least one herpesvirus, preferably by measuring DNAemia, and
d) establishing the presence of an increased risk of complications when said viral load determined for said sample is greater than the predetermined reference value and when at least one herpesvirus is present.

According to another preferred embodiment, the description relates to an in vitro or ex vivo method for evaluating the risk of complications in a patient who is not taking an immunosuppressive treatment and who has been admitted to a healthcare facility, comprising measuring the viral load of at least one torque teno virus (TTV) in a biological sample from said patient, and further comprising measuring the viral load of at least one herpesvirus in said biological sample from the patient.

In the present case, “viral load of at least one HPV” or “load of at least one HPV” corresponds to the viral load of at least one HPV strain, i.e. to the number of viral particles of at least one HPV strain which are present in a biological sample.

The load of at least one HPV in a patient means the viral load of at least one HPV strain which said patient is hosting. The HPV load can be determined by measuring the amount of a component of at least one HPV strain, such as a nucleic acid or a protein, in this biological sample. Preferably, the HPV load corresponds to the amount of nucleic acid sequences of a given HPV strain present in a biological sample.

Thus, according to the invention, it is possible to differentiate between the different specific HPV strains to be measured in said biological sample. Preferably, the determination of the HPV load comprises the determination of the amount of active and/or inactive virus copies. It consists in determining the amount of circulating, integrated or latent virus copies.

Methods for detecting and/or measuring the viral load of an HPV nucleic acid in a biological sample are the same as those described previously for TTV and comprise, inter alia, amplification, preferably amplification by PCR, more preferably by real-time PCR, sequencing, hybridization with a labeled probe, and all other methods known to those skilled in the art.

Herpes viruses (Herpesviridae or HpV) are well known to those skilled in the art. They are a family of DNA viruses that affect various animal species including birds, fish, reptiles, amphibians and mammals, including humans. All the members of the HpV family share a common structure: a relatively large, monopartite, double-stranded, linear DNA encoding 100 to 200 genes encased within an icosahedral protein cage called the capsid, which is itself wrapped in a protein layer called the tegument containing both viral proteins and viral mRNAs and a lipid bilayer membrane called the envelope. All HpVs are nuclear-replicating; the viral DNA is transcribed to mRNA within the infected cell's nucleus, where it can remain in a latent form indefinitely.

Nine types of HpV are known to chiefly infect humans. At least five are extremely widespread in humans: herpes simplex 1 and 2 (HSV-1 and HSV-2, also known as HHV-1 and HHV-2), both of which can cause orolabial and genital herpes; varicella zoster (or HHV-3), the cause of chickenpox and shingles; Epstein-Barr virus (EBV or HHV-4) implicated in several diseases, including mononucleosis and some cancers; and human cytomegalovirus (HCMV or CMV or HHV-5).

More than 90% of adult humans have been infected with at least one of these viruses, and a latent form of the virus remains in almost all humans who have been infected. Less common human herpesviruses are human herpesvirus 6A and 6B (HHV-6A and HHV-6B, together referred to as HHV-6), human herpesvirus 7 (HHV-7), and Kaposi's sarcoma-associated herpesvirus (KSHV, also known as HHV-8).

“Herpes virus” or “HPV” or “HHV” (Human Herpes Virus) here mean a virus of the Herpesviridae family. “HPV genome” refers here to the genomes of all the Herpesviridae, including those of the type HSV-1, HSV-2, HHV-3, EBV, CMV, HHV-6, HHV-7 and KSHV, and more particularly those of the type CMV, EBV, HHV6 and HSV-1.

According to a preferred embodiment, said at least one herpesvirus is selected from the group consisting of: CMV, EBV, HHV6 and HSV-1. Preferably, the herpesvirus is EBV. By way of example, reference is made here to the genome of herpesvirus strains of the type CMV AD169, EBV B-95, HSV1 95 and HHV6 Z29. By way of nonlimiting example, a genome of CMV AD169 may be that for which the sequence is referenced under Genbank accession number X17403.1. Similarly, a genome of EBV B-95 may be that for which the sequence is referenced under Genbank accession number V01555.2. An example genome of HHV6 Z29 may be that for which the sequence is referenced under Genbank accession number X83413.2.

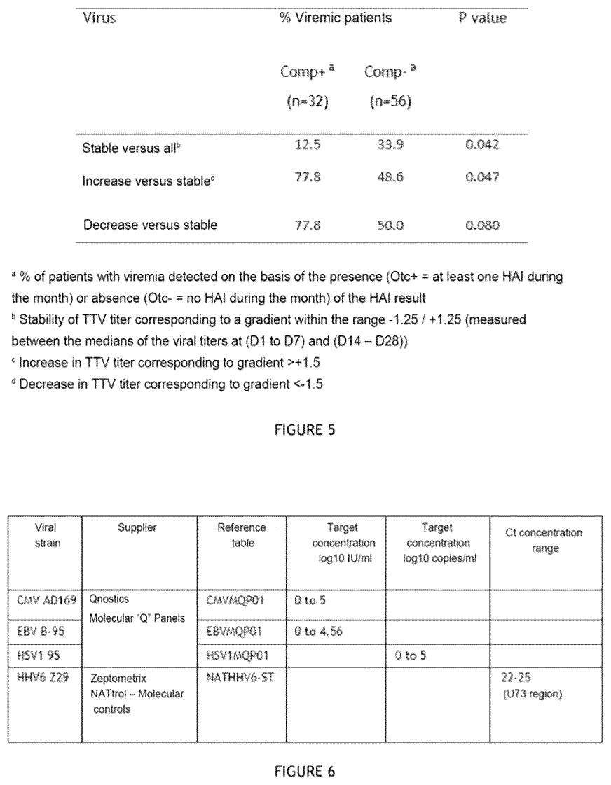

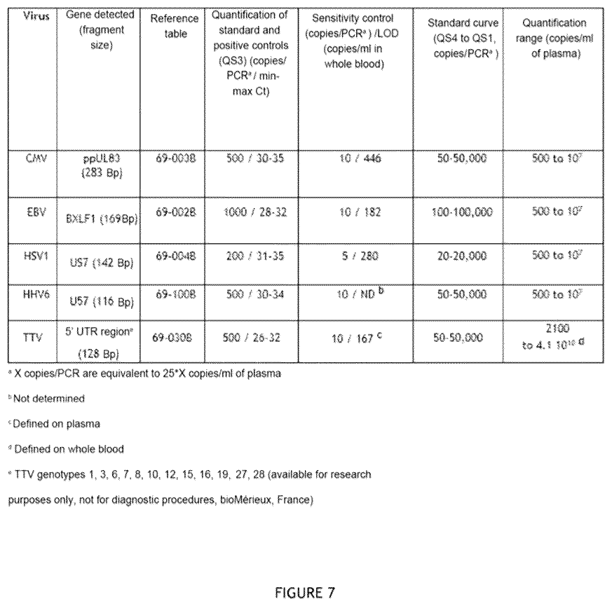

Preferentially, the presence of at least one HPV is determined, and/or the HPV viral load is measured, using real-time quantitative PCR. Numerous methods for detecting and quantifying HPVs have been described in the art (see for example Walton et al., Plos ONE 9(6): e98819, 2014). Reference will particularly be made to the method described by Mallet et al. (Intensive Care Medicine Exp. (2019) 7:28) and more particularly in table S1 of this article, reproduced here in FIGS. 6 and 7. This method is particularly advantageous because of its simplicity and robustness; it may also be implemented using any type of PCR platform. It is based on the amplification of genes specific to each of these herpesviruses, namely ppUL83 which encodes a structural protein of CMV, the gene BXLF1 which encodes a thymidine kinase of EBV, the gene US7 which encodes a glycoprotein of HSV-1, and the gene U57 which encodes a capsid protein of HHV6. It is particularly advantageous to use the R-GENE® diagnostic kits (CMV R-GENE® Ref. 69-003B, EBV R-GENE® Ref.: 69-002B, HSV1 HSV2 VZV R-GENE® Ref.: 69-004B and HVV6 R-GENE® Ref.: 69-100B, bioMérieux, France) to implement this method.

In a preferred embodiment, the method described herein comprises an additional step of standardizing the amount of HPV nucleic acid measured. To this end, those skilled in the art can refer to the methods and embodiments described above regarding the standardization of TTV levels. These methods can be readily adapted to the standardization of the amount of HPV nucleic acid.

Once the load of at least one HPV is determined by measuring the amount of HPV nucleic acid, the latter optionally being standardized, it may be advantageous to compare it to a reference HPV load. To this end, those skilled in the art can refer to the methods and embodiments described above regarding the comparison to TTV reference levels. These methods can be readily adapted to the comparison to reference HPV levels.

### Methods for Treating a Patient Suspected of Having a Risk of Complications

The susceptibility of the patient to risks of complications, particularly of infections, can therefore be determined using the methods described herein, which makes it possible to develop and adapt a treatment specific to the patient's needs.

Advantageously, the methods as described previously can therefore comprise, in all embodiments thereof, a step of managing healthcare, particularly in order to reduce the risk of complications and in particular the risk of occurrence of a healthcare-associated infection.

A patient identified as having an increased risk of occurrence of a healthcare-associated infection may have suitable managed healthcare with the aim of reducing the risk of occurrence of said healthcare-associated infection and, for example, to reduce the risk of developing sepsis, septic shock or else the risk of death.

Thus, according to another aspect, the present invention also relates to a method for treating a patient suspected of having a risk of complications, characterized in that it comprises the steps consisting in:
identifying patients at risk of complications by implementing any one of the embodiments described above, and
adapting management of the healthcare of said patient identified in the preceding step in order to reduce the risk of worsening.

By way of examples of managing the healthcare, mention may be made of an immunomodulatory treatment adapted to the patient or else a prophylactic antibiotic treatment, it being possible for both treatments to be combined, and/or turning to a high-dependency unit or resuscitation department in order to reduce the risk of occurrence of a healthcare-associated infection, for example reduce the risk of developing sepsis, septic shock or even the risk of death, in the days following the measurement of the expression of the biomarker(s). Preferably, the immunomodulatory treatment is an immunostimulatory treatment if it is determined that the individual has an immunosuppressed status, or an anti-inflammatory treatment if it is determined that the individual has an inflammatory status.

Among the immunostimulatory treatments which may be chosen, mention may be made by way of example of the interleukin group, in particular IL-7, IL-15 or IL-3, growth factors, in particular GM-CSF, interferons, in particular IFNγ, toll-like receptor agonists, in particular antibodies directed against immune checkpoints (for example anti-PD1, anti-PDL1, anti-LAG3, anti-TIM3, anti-IL-10 or anti-CTLA4 antibodies), transferrins and apoptosis-inhibiting molecules, FLT3L, thymosin al and adrenergic antagonists.

Among the anti-inflammatory treatments, mention may particularly be made of the glucocorticoids group, cytostatic agents, molecules acting on immunophilins and cytokines, molecules that block the IL-1 receptor, and anti-TNF treatments. Examples of suitable prophylactic antibiotic treatments for preventing pneumonia are described in particular in the Annales Francaises d'Anesthésie et de Réanimation (30; 2011; 168-190). Conversely, it will be possible to quickly send a patient without the risk of occurrence of a healthcare-associated infection to an outpatient department, for example an infectious disease department, rather than remaining in a department with close monitoring which they do not require.

The invention will be described more precisely by means of the examples below.

### Examples

In order to better understand the physiopathology underlying virus reactivation phenomena in patients admitted to the intensive care unit, a study was carried out in the cohort of patients involved in the REALISM clinical study (NCT01931956). For each of these patients, the blood DNAemia of the TTV anellovirus and of the herpesviruses CMV, EBV, HHV6, HSV-1 was measured over a period of one month following their admission to intensive care. The aim of the expected results was to make it possible to identify common complications associated with virus reactivation phenomena in these patients and to identify whether one or more viruses might be associated with the occurrence of particular complications.

### Materials and Methods

### Population of the Study

An observational study was carried out on prospective cohorts of seriously ill patients presenting either with sepsis, severe trauma, severe burns or having undergone planned surgery in the anesthesiology and intensive medicine department of the Edouard Herriot hospital (Hospices Civils de Lyon, France). The inclusion period was 28 months (December 2015-March 2018). The study protocol was approved by the institutional ethics committee (Comite de Protection des Personnes Sud-Est//) under number 2015-42-2. This clinical study was also registered at clinicaltrials.gov (NCT02638779). At the time of inclusion, written informed consent was obtained from each healthy volunteer and each patient. When a patient was not able to directly consent, informed consent was obtained from the patient's legal representative and reconfirmed with the patient at the earliest possible occasion.

The inclusion criteria were as follows: patients aged over 18, clinical diagnosis of septicemia as defined by the SEPSIS-3 consensus definitions of 2016 (Singer et al., 2016), severe trauma with an injury severity score (ISS)>15, severe burns with a total burn surface area of greater than 30%, or surgical patients who have to undergo major surgery such as esophagogastrectomy, Bricker bladder resection, cephalic pancreaticoduodenectomy and/or abdominal aortic aneurysm surgery by laparotomy.

The exclusion criteria were as follows: presence of a pre-existing disorder or treatment that might influence the patients' immune status; in particular, patients under immunosuppressive treatment, pregnant patients, institutionalized patients and those for whom informed consent could not be obtained were excluded.

In parallel, a cohort of 175 healthy volunteers aged from 18 to 82 years (81 men and 94 women) were prospectively recruited. In order to take into account the possible influence of age and sex on immune parameters, the distribution of the healthy volunteers was based on demographic data regarding age and sex of the population of France in 2016.

### Sampling and Data Collection

For each patient, the samples and clinical data were collected three times during the first week after admission: (1) on day 1 or 2 (D1-2), (2) on D3 or 4 (D3-4) and (3) on D5, D6 or D7 (D5-7), then twice more during the month following admission, (4) on D14 and (5) on D28. For the healthy volunteers, a sample was taken during the study visit and the clinical data was recorded. The data collection is described in detail elsewhere (Venet et al., 2021).

The demographic data for the patients, comorbidities, diagnosis, severity and clinical results were prospectively recorded manually. Longitudinal monitoring was performed for a period of 30 days. In each case, whole peripheral blood taken from each patient and from each healthy volunteer was processed within the 3 hours following the sample being taken.

The blood was collected in an EDTA tube for determining the plasma viral DNAemia, immune phenotyping by flow cytometry and measurements of plasma cytokine levels, in two heparin tubes for the functional tests (proliferation, cytokine production experiments), and finally in a blood RNA PAXgene tube (PreAnalytix, Hilden, Germany) for the measurements of the biomarker mRNA concentration in whole blood by real-time PCR using EvaGreen or TaqMan probe methodologies.

The PAXgene samples were stabilized for at least 2 h at ambient temperature after sampling, then frozen at −80° C. according to the manufacturer's recommendations.

### Definitions of Complications

The main complications evaluated in the context of this study were secondary infection (healthcare-associated infection—HAI) at 28 days (D28) and mortality at D28. The other complications evaluated included the length of the stay in the intensive care unit (ICU) and the total length of the hospital stay, the daily use of an invasive medical device (tracheal intubation, indwelling urinary catheter and central venous line). The information collected regarding the infections was examined by an independent decision-making committee composed of three clinicians who were not involved in recruiting or caring for the patients in the study.

The confirmation of the diagnosis of a secondary infection (healthcare-associated infection, HAI) by this committee is based on the guidelines from the European Society of Clinical Microbiology and Infectious Diseases and from the Infectious Diseases Society of America. Only the first episode of secondary infection was taken into account in the analyses.

### Determination of Viral DNAemia

The semi-automated process for determining viral DNAemia of TTV and four herpesviruses, integrating processing of the sample and standardized procedures for real-time quantitative PCR, has been previously described in detail (Mallet et al., 2019—Intensive Care Medicine Exp. 7:28).

Briefly, viral DNA was extracted from the plasma samples using the Maxwell® HT Viral TNA chemistry kit (Promega) composed of paramagnetic silica particles and the Freedom EVO® liquid handling robot (TECAN), following the manufacturer's instructions. PCR controls were added to each plasma sample before extraction. The limit of detection (LOD) was determined beforehand for the semi-automated process to be the smallest amount that can be distinguished from an absence of detection (blank value) using viral standards (Mallet, 2019—Intensive Care Medicine Exp. 7:28).

The limit of detection of TTV was determined using plasma samples comprising a predetermined amount of TTV. Firstly, the control plasma sample is serially diluted from 10⁻¹ to 10⁻⁴, and each diluted sample is extracted independently 3 times then amplified using quantitative PCR in order to determine an approximate limit of detection. Secondly, the greatest dilution that gives a positive signal is again diluted to 1/2 and to 1/4, then each diluted sample is extracted independently 20 times and amplified. The most diluted positive replicates with 100% detection are used as the limit of detection.

The LOD, expressed as copies per ml of plasma, is 167 for TTV, 100 for CMV, 33 for EBV, 166 for HHV6, and 33 for HSV1. The viral DNAs were amplified using real-time PCR on the StepOnePlus™ Real-Time PCR system (ThermoFisher SCIENTIFIC) by means of R-GENE® assay kits using TaqMan probes for CMV, EBV, HSV1, HHV6 and TTV (bioMérieux SA).

All the nucleic acid samples, randomly distributed in the plates, were amplified simultaneously with the quantification standards, sensitivity controls and negative controls, in accordance with the manufacturer's instructions.

Finally, using standard curves, the Ct (“cycle threshold”) values for each sample were converted into copies/μl of viral DNA in the PCR reaction tube, then into copies/ml of viral DNA in the plasma samples.

### Statistical Analysis

Viremia was measured by measuring the DNAemia for each virus, with a viremic event, i.e. positive DNAemia, being characterized by a number of copies of the virus per microliter that is greater than a predefined threshold (greater than the LOD). A positive DNAemia is considered to be early if it is detected for at least one of the samples from the patient during the first week (day 1/2, day 3/4 or day 5/7) after the patient is admitted to intensive care. DNAemia was also evaluated on D14 and D28. The data is presented in the form of numbers and percentages (qualitative variables), and medians and 25th/75th percentiles (quantitative variables).

The association between a viremic event and clinical complications (survival or bacterial infection acquired in intensive care, as described previously (Mallet et al., 2019—Intensive Care Medicine Exp. 7:28)) was evaluated using the chi squared test or Fisher's exact test, where appropriate.

The binary association between DNAemia/a viremic event and the quantitative immune markers (cells, cytokines, mRNA) was evaluated using the Wilcoxon signed-rank test. Finally, analyses were performed using the R software, version 3.4.4, and statistical significance was defined as an alpha risk of incorrectly rejecting the null hypothesis of 5% (p<0.05 indicates a significant association).

### Results

### Description of the Cohort

The REALISM cohort of 377 seriously ill patients is composed of: 107 cases of septicemia, 137 cases of severe trauma, 24 cases of severe burns and 109 cases of major surgery.

Quantitative data (viral load) rather than qualitative data (DNAemia) may enable better discrimination between (1) a non-significant viral load, (2) virus “reactivation” as a putative marker of immunosuppression, and (3) high viral loads supporting the presence of a real viral infection requiring treatment (Textoris et al., 2017).

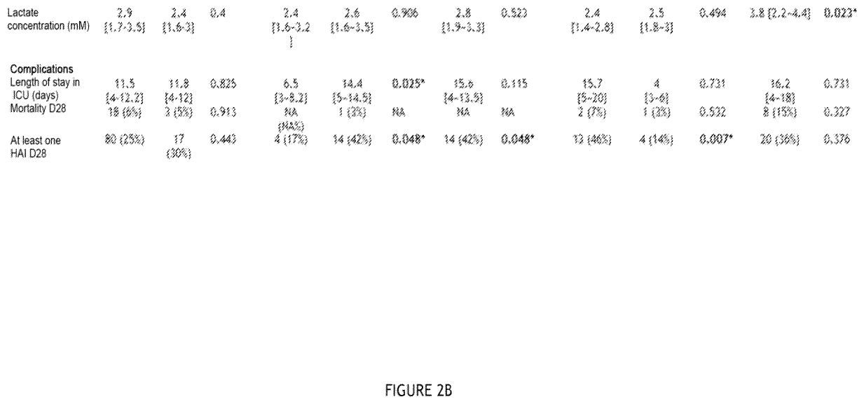

Patients with high TTV DNAemia (viral load greater than 10 000 copies/ml) were more seriously ill, with higher severity scores and an increased need for treatment (FIG. 3).

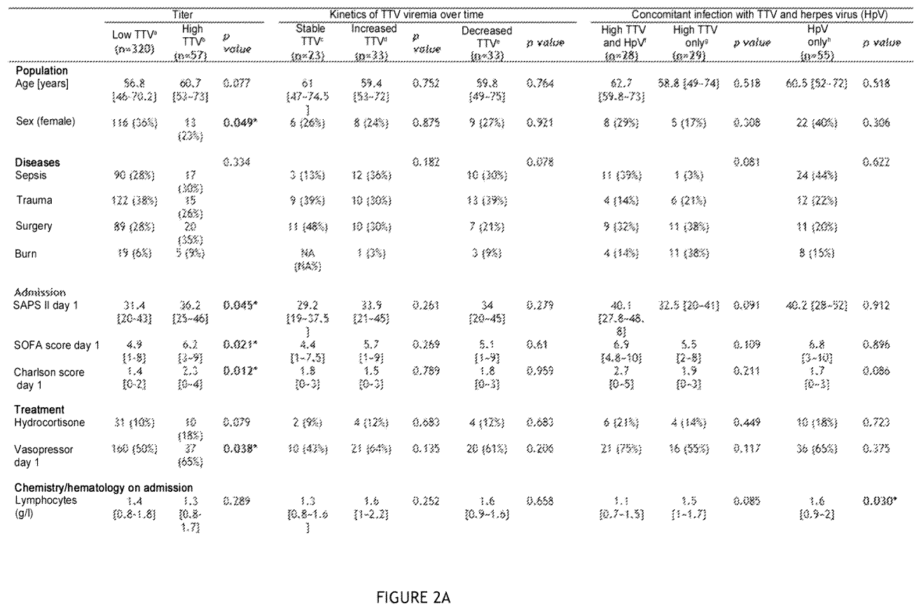

While the increase in the TTV viral load (p=0.025) was significantly correlated with a longer stay in intensive care, both an increase (p=0.048) and a decrease (p=0.048) in the TTV viral load, in other words a variation in the TTV viral load, were significantly associated with the occurrence of a healthcare-associated infection acquired in intensive care during the first month (FIGS. 2A and 2B).

Moreover, it is surprising to observe that cases of HAIs tend to be more common in patients with positive DNAemia for TTV and at least one herpesvirus than in patients just with positive herpes DNAemia (FIGS. 2A and 2B).

### DNAemia in Patients in the Intensive Care Unit During the First Week and First Month of Hospitalization

### During the First Week

TTV was detected (positive DNAemia) in 217 (58%) of the patients in the cohort, namely slightly more than in the healthy controls (51%). TTV was detected alone in 160 (42%) patients, and in the patients was detected concomitantly with 1 (n=48, 13%) or 2 (n=6, 2%), or 3 and more (n=3, 1%) herpesviruses.

The most common herpesvirus concomitantly detected with TTV was EBV (n=29, 8%), followed by HHV6 (n=18, 5%), HSV1 (n=12, 3%) and CMV (n=12, 3%). Overall, the number of patients with positive DNAemia for a single virus (n=189), whether that is a herpesvirus or TTV, was similar to the number of patients with positive DNAemia for the presence of at least two viruses (n=188).

### During the First Month

TTV was detected in 228 (60%) patients of the cohort. 151 (40%) patients had positive DNAemia for TTV only, while TTV was concomitantly detected (positive DNAemia) in the patients with 1 (n=59, 16%), 2 (n=11, 3%) or 3 or more (n=7, 2%) herpesviruses. All the herpesviruses were concomitantly detected with TTV, with the most common concomitant detection being EBV (n=36, 10%), then HSV1 (n=28, 7%), CMV (n=23, 6%) and HHV6 (n=19, 5%). Overall, the cohort comprised as many patients with an infection (positive DNAemia) from a single virus (n=187) as patients having concomitant infection (n=190).

The percentage of patients with a TTV was similar in patients suffering from septicemia, severe trauma and major surgery (56 to 64%) and slightly lower in burns victims (46%), compared to 51% observed in the healthy volunteers.

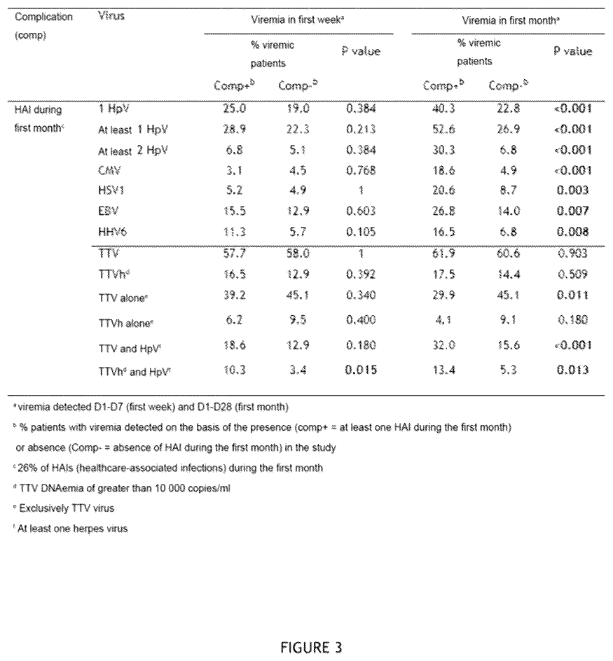

As a trend, the percentage of patients with a high TTV titer appeared to be larger in the group of patients who had undergone surgery, and the severity criteria are correlated with a high TTV viral load (in particular, greater than 7000 copies/ml) (FIG. 4).

Association of DNAemia with Clinical Complications

Considering at the end of the 28 day observation period, the occurrence of at least one episode of a secondary, healthcare-associated infection.

The detection of each herpesvirus individually over the first week of admission is not significantly associated with a risk of healthcare-associated infection (HAI), unlike the first month. A significant increase in the rate of HAIs is observed in patients who had, during the first month, at least one viremic episode (positive DNAemia) for any herpesvirus considered individually, in the absence of positive DNAemia for TTV.

In addition, positive DNAemia for TTV alone (without detection of a herpesvirus) is associated with a reduced occurrence of HAI (FIG. 3).

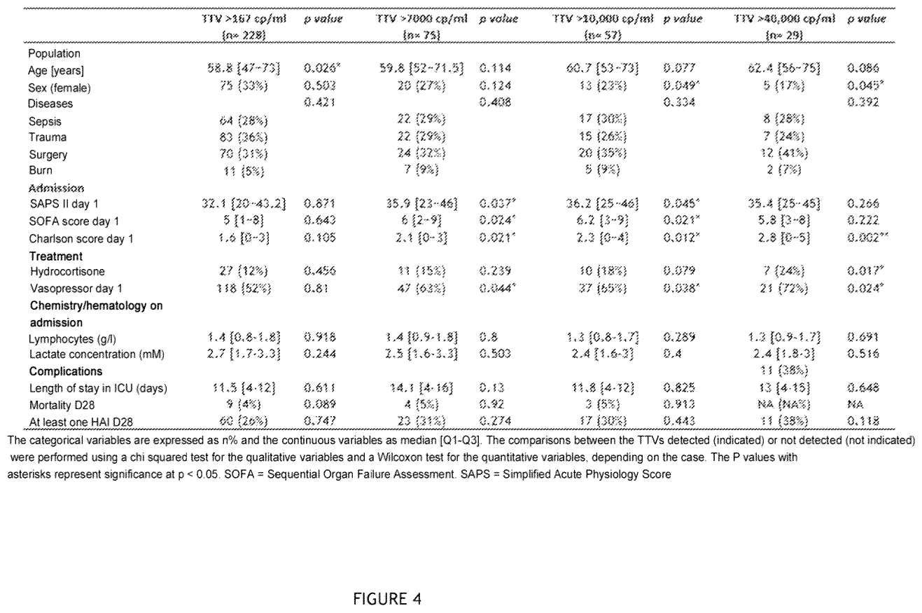

It was also observed that a stable TTV viral load over the first month is associated with a reduced occurrence of HAI (FIG. 5).

If the viral DNAemia of the patients from the cohort over the first month is considered, in those patients who had several viremic events (positive DNAemia for 2 or more herpesviruses, or for a TTV and at least one herpesvirus) a higher rate of HAI (44%) is observed than in those patients who had positive DNAemia for a single virus (22%) or patients without detected DNAemia (19%) (chi squared test, p<0.001).

If the events of positive viral DNAemia over the first week are considered, those patients who had several viremic events have a similar rate of HAI (11%) to those who had positive DNAemia for a single virus (9%) or without any detected DNAemia (9%) (chi squared test, P=0.8).

Surprisingly, the results show that, in an early manner, as early as the first week following the patient's admission, the concomitant detection of a high TTV viral load (in particular greater than 10 000 copies/ml of sample) and a positive DNAemia for a herpesvirus is significantly associated with a risk of occurrence of a healthcare-associated infection (FIG. 3).

In summary, in terms of viremia, the disease group and the admission criteria, (i) the proportion of patients with a variable (increasing or decreasing) TTV viral load over time tends to be higher in patients suffering from septicemia than for the other groups, and (ii) positive DNAemia for herpes as well as a high TTV viral load appear to be associated with the severity of the patient's condition on admission.

In terms of HAIs, (i) the concomitant detection, preferably as early as the first week of admission, of a high TTV viral load and positive DNAemia for herpes is correlated with the risk of more frequent occurrence of HAI, and (ii) a stable TTV viral load is associated with a relatively low occurrence of HAI, unlike a variation in the TTV viral load which is also correlated with the risk of occurrence of HAI in the patient.

### Discussion

The results demonstrate that it is possible to distinguish a non-significant TTV load from a high TTV load. Thus, it is to the inventors' credit in particular to have identified a TTV expression threshold associated with physiopathological characteristics in patients in intensive care.

In short, the results presented previously (i) demonstrated that the detection in the patient, as early as the first week of admission to intensive care, of a high TTV load associated with herpesvirus reactivation, was associated with an increased risk of HAI, and that (ii) early and high TTV viremia was associated with a compromised immune system.

The change in viremia over time was less discernible for TTV (from 58% in the first week to 60% over the month), and positive viremia for TTV was more frequent for TTV alone than for concomitant detection of TTV and herpesvirus (42% versus 15% measured during the first week, 40% versus 20% measured during the first month).

While the prevalence of TTV is higher in men in the cohorts of individuals in good health (Focosi et al., 2020) and transplant recipients (Bal et al., 2018), no gender bias was observed in the population of this study, except for a predominance of men in the sub-group with TTV viral loads of greater than 10 000 copies/ml.

TTV and EBV were the viruses most frequently detected concomitantly, from 8% to 10% during the first week and the first month, respectively.

During the same period, only variations in the TTV load are significantly associated with an increased risk of occurrence of HAI. Indeed, the presence of TTV alone, even at a high load, is insufficient to determine an increased risk of HAI.

Nevertheless, during the first month following admission, patients with multiple viremic events, including those with positive DNAemia for TTV, have a higher frequency of HAI (44%) than those who had a single viremic event or no viremic events (22% and 19%, respectively). Thus, entirely surprisingly and unexpectedly, the concomitant detection of a high TTV load and a herpesvirus during the first week of admission is significantly associated with the occurrence of a HAI event during the first month.

In conclusion, the results demonstrate, in patients admitted to the intensive care unit, the existence of a correlation between virus reactivation, in particular TTV, combined or not combined with HpV, and the risk of developing healthcare-associated infections.

### REFERENCES

1. Nishizawa T, Okamoto H, Konishi K, Yoshizawa H, Miyakawa Y, Mayumi M. A Novel DNA Virus (TTV) Associated with Elevated Transaminase Levels in Posttransfusion Hepatitis of Unknown Etiology. Biochem Biophys Res Commun. December 1997; 241(1):92-7.
2. Focosi D, Antonelli G, Pistello M, Maggi F. Torquetenovirus: the human virome from bench to bedside. Clin Microbiol Infect. July 2016; 22(7):589-93.
3. Spandole S, Cimponeriu D, Berca L M, Mihaescu G. Human anelloviruses: an update of molecular, epidemiological and clinical aspects. Arch Virol. April 2015; 160(4):893-908.
4. de Villiers E-M, Borkosky S S, Kimmel R, Gunst K, Fei J-W. The diversity of torque teno viruses: in vitro replication leads to the formation of additional replication-competent subviral molecules. J Virol. July 2011; 85(14):7284-95.
5. Kosulin K, Kernbichler S, Pichler H, Lawitschka A, Geyeregger R, Witt V, et al. Post-transplant Replication of Torque Teno Virus in Granulocytes. Front Microbiol. 2018; 9:2956.
6. Maggi F, Bendinelli M. Immunobiology of the Torque Teno Viruses and Other Anelloviruses. In: de Villiers E-M, Hausen H zur, editors. TT Viruses [Internet]. Berlin, Heidelberg: Springer Berlin Heidelberg; 2009 [cited on 20 Aug. 2019]. p. 65-90. Retrieved from: http://link.springer.com/10.1007/978-3-540-70972-5_5
7. Hino S, Miyata H. Torque teno virus (TTV): current status. Rev Med Virol. February 2007; 17(1):45-57.

1. A method for the in vitro or ex vivo evaluation of the risk of complications in a patient admitted to a healthcare facility and who is not undergoing immunosuppressive treatment, characterized in that it comprises carrying out the steps consisting in:
a) measuring the viral load of at least one Torque Teno Virus (TTV) in a biological sample from said patient,
b) comparing the viral load determined for said biological sample to a predetermined reference value, and
c) establishing a risk of complications when the viral load determined for said biological sample is greater than or less than the predetermined reference value.

2. The method as claimed in claim 1, characterized in that the predetermined reference value is 10 000 copies/ml.

3. The method as claimed in claim 2, characterized in that the presence of an increased risk of complications is established when said viral load determined for said sample is greater than said reference value.

4. The method as claimed in claim 1, characterized in that it also comprises detecting the presence of at least one herpesvirus, preferably by measuring DNAemia.

5. The method as claimed in claim 4, characterized in that the presence of an increased risk of complications is established when the viral load of at least one TTV determined for said sample is greater than 10 000 copies/ml and when at least one herpesvirus is present.

6. The method as claimed in claim 1, characterized in that it comprises carrying out the steps consisting in:
a) measuring a first viral load of at least one TTV in said biological sample from the patient originating from a first sample taken at the time T1,
b) measuring a second viral load of at least one TTV in said biological sample from the patient originating from a second sample taken at the time T2,
c) calculating the variation between the viral load at T2 and the viral load at T1, giving a ΔTTV value,
d) establishing a conclusion regarding the risk of complications from the result of the comparisons.

7. The method as claimed in claim 6, characterized in that the conclusion is drawn that an increased risk of complications is absent when the ΔTTV value is within the range extending from −1.25 to +1.25, preferably from −1.20 to +1.20, and more preferably still from −1.10 to +1.10.

8. The method as claimed in claim 1, characterized in that the risk of complications is the risk of a healthcare-associated infection (HAI) occurring.

9. The method as claimed in claim 1, characterized in that the biological sample originates from a patient admitted to hospital, preferably to the emergency department, the resuscitation department, the intensive care unit or the high-dependency unit.

10. The method as claimed in claim 1, characterized in that the biological sample originates from a patient in a septic state, more particularly in septic shock, a patient suffering from burns, more particularly severe burns, a patient suffering from trauma, more particularly severe trauma, or a patient who has undergone a surgical operation, more particularly a major surgical operation.

11. The method as claimed in claim 1, characterized in that the biological sample originates from a patient in a septic state, suffering from burns, suffering from trauma, or who has undergone a surgical operation, and who has been admitted to the intensive care unit.

12. The method as claimed in claim 4, characterized in that said at least one herpesvirus is selected from the group consisting of: CMV, EBV, HHV6 and HSV-1, and is preferably EBV.

13. The method as claimed in claim 1, characterized in that the biological sample is a biological fluid from the patient, selected from the group consisting of blood or derivatives thereof, such as plasma and/or serum, cerebrospinal fluid, urine, and bronchoalveolar lavage.
